(19) United States
(12) Patent Application Publication
(10) Pub. No.: US 2023/0287350 A1
(43) Pub. Date: Sep. 14, 2023

(54) PRODUCTION OF EXTRACELLULAR VESICLES FROM MUSCLE CELLS

(71) Applicant: TECHNION RESEARCH AND DEVELOPMENT FOUNDATION LTD, Haifa (IL)

(72) Inventors: Shulamit Levenberg, Haifa (IL); Shaowei Guo, Haifa (IL); Lior Debbi, Haifa (IL)

(21) Appl. No.: 18/005,879

(22) PCT Filed: Aug. 16, 2021
(86) PCT No.: PCT/IL2021/050994

§ 371 Date: Jan. 18, 2023

Related U.S. Application Data
(60) Provisional application No. 63/067,347, filed on Aug. 19, 2020.

(51) Int. Cl. C12N 5/077; C12M 1/42; C12M 1/12

(52) U.S. Cl. CPC C12N 5/0658; C12M 35/04; C12M 25/14; C12N 2513/00

(57) ABSTRACT

The present invention provides methods and systems for enhanced production and/or secretion of extracellular vesicles (EVs) from muscle cells utilizing various dynamic mechanical loading profiles thereon, cultured on three-dimensional (3D) scaffolds. The scaffolds may comprise a plurality of layers, wherein each layer comprises a plurality of elastic microfibers, and wherein the microfibers are aligned in parallel to a longitudinal axis and to each other. The elastic 3D scaffold may be configured to undergo dynamic mechanical loading profiles and support an expansion of a population of muscle cells cultured thereon into a 3D multi-layer structure of muscle cells, wherein said 3D multi-layer structure is configured to produce and/or secret extracellular vesicles into a medium.

### FIELD OF THE INVENTION

Provided herein are systems and methods for enhanced secretion of extracellular vesicles from muscle cells cultured on elastic three-dimensional scaffolds.

### BACKGROUND OF THE INVENTION

Regeneration of skeletal muscle or cardiac muscle in the body is limited. Although regeneration can occur after minor injuries, major injuries can result in irreversible damage to muscle, leading to scarring, fibrosis and even loss of muscle function (J. S. Choi et al., “Exosomes from differentiating human skeletal muscle cells trigger myogenesis of stem cells and provide biochemical cues for skeletal muscle regeneration”, J Control Release 222 (2016) 107-15). The current mainstream solution for repairing scarred tissue is reconstructive surgery (J. M. Grasman, et al., “Biomimetic scaffolds for regeneration of volumetric muscle loss in skeletal muscle injuries”, Acta Biomater 25 (2015) 2-15). However, this approach is invasive and fails to address critical requirements for clinical use. Cell therapy has been tested in preclinical studies, showing the promise of accelerating muscle regeneration (P. von Roth, et al., “Mesenchymal stem cell therapy following muscle trauma leads to improved muscular regeneration in both male and female rats”, Gend Med 9(2) (2012) 129-36). However, the paracrine role of cells in tissue regeneration is attracting particular attention.

Extracellular vesicles (EVs), including exosomes, are nanometer vesicles secreted from cell membrane, carrying various cargos, including genetic materials, proteins and lipids, for conducting intercellular communications (G. van Niel, et al., “Shedding light on the cell biology of extracellular vesicles”, Nat Rev Mol Cell Biol 19(4) (2018) 213-228).

Several studies showed the beneficial effects of exosomes-based therapies in skeletal and cardiac muscle regeneration using both in vitro and in vivo models (Y. Nakamura, et al., “Mesenchymal-stem-cell-derived exosomes accelerate skeletal muscle regeneration”, FEBS Lett 589(11) (2015) 1257-65, and B. Liu, et al., “Cardiac recovery via extended cell-free delivery of extracellular vesicles secreted by cardiomyocytes derived from induced pluripotent stem cells”, Nat Biomed Eng 2(5) (2018) 293-303).

In vitro, exosomes-treated myoblasts expressed faster and higher differentiation rates, and exosomes-treated endothelial cells had an increase in vessel formation (M. Guescini, et al., “Extracellular Vesicles Released by Oxidatively Injured or Intact C2C12 Myotubes Promote Distinct Responses Converging toward Myogenesis”, Int J Mol Sci 18(11) (2017), and S. M. Davidson, et al., “Endothelial cells release cardioprotective exosomes that may contribute to ischaemic preconditioning”, Sci Rep 8(1) (2018) 15885). Subsequently, in-vivo models of treatments by exosomes showed significant tissue regeneration after skeletal muscle injury or cardiac infraction.

Rome, Sophie, et al. review the production of extracellular vesicles (EVs) from muscle cells, which are mainly based on 2D static cultivation of cells (“Skeletal muscle-released extracellular vesicles: State of the art.” Frontiers in physiology 10 (2019): 929).

In order to produce extracellular vesicle (such as exosomes) from various cells, bioreactors systems can be used. For example, D. B. Patel et al. discloses the use of a 3D-printed scaffold-perfusion bioreactor system to assess the response of dynamic culture on extracellular vesicle production from endothelial cells (ECs) (D. B. Patel, et al., “Enhanced extracellular vesicle production and ethanol-mediated vascularization bioactivity via a 3D-printed scaffold-perfusion bioreactor system,” Acta Biomater., pp. 1-9, 2018).

Watson, D. C et al. discloses a hollow-fiber bioreactor for the efficient production of bioactive extracellular vesicles bearing the heterodimeric cytokine complex Interleukin-15:Interleukin-15 receptor alpha (Watson, D. C., et al., (2016). “Efficient production and enhanced tumor delivery of engineered extracellular vesicles”. Biomaterials, 105, 195-205).

Lovecchio, J et al. discloses a prototype standalone perfusion/compression bioreactor system for dynamic compression of stem cells seeded onboard of 3D chitosan-graphene (CHT/G) templates (Lovecchio, J., Gargiulo, P., Vargas Luna, J. L. et al. “A standalone bioreactor system to deliver compressive load under perfusion flow to hBMSC-seeded 3D chitosan-graphene templates”. Sci Rep 9, 16854 (2019)).

International Pub. No. WO2020/0261257 discloses methods and systems for enhanced production and/or secretion of extracellular vesicles from at least one three-dimensional porous scaffold having a population of stem cells cultured thereon, utilizing various shear stress conditions on a variety of stem cells.

However, there are several drawbacks associated with the previously known methods. The common protocols for exosomes production are mainly based on 2D static platforms and are not adjusted specifically to muscle tissue and therefore have limited production yields. There remains an unmet need for simple and cost-efficient methods and systems for inducing advanced secretion of extracellular vesicles from muscle cells cultured on three-dimensional scaffolds.

### SUMMARY OF THE INVENTION

The present invention provides systems and methods for inducing or increasing production and/or secretion of extracellular vesicles (EVs) from muscle cells. Muscle cells include but not limited to, skeletal muscle cells, smooth muscle cells and induced pluripotent stem cells derived-cardiomyocytes. The methods of the present invention comprise culturing the cells on an elastic three-dimensional (3D) scaffold with favorable fiber orientation, optionally within a bioreactor system, and inducing various dynamic mechanical loading profiles on the 3D scaffold and the cells.

Since the previously known protocols for production of EVs from muscle cells are mainly based on 2D static cultivation of cells, they can only provide limited yields. Advantageously, the dynamic mechanical loading profiles provided in the present invention induce physiological changes in the cells, that result in enhanced secretion of EVs, and in some embodiments result in modified EV properties and improved biological effect of the EVs on muscle cells. The present inventors have discovered that culturing myoblasts or muscle cells on a stretchable and elastic 3D scaffold with a favorable/specific fiber orientation can improve attachment, proliferation, and differentiation towards mature muscle cells therefrom. Furthermore, providing cyclic mechanical tension stimuli (e.g., stretching) thereto, along and/or in parallel to the specific fiber orientation within the scaffold, can significantly improve exosome production rates as compared to static/un-stretched conditions (e.g., an 11-fold higher in yield). It is contemplated that muscle-based EVs produced under the conditions of the present invention hold the potential to provide enhanced and improved treatments for various muscle injuries (such as skeletal or cardiac).

According to a certain aspect, there is provided a method for producing extracellular vesicles (EVs) from muscle cells, the method comprising the steps of: (a) providing at least one three-dimensional (3D) scaffold comprising: a plurality of layers, wherein each layer comprises a plurality of elastic microfibers spaced apart from each other, wherein each microfiber extends from a first end of the scaffold towards a second end of the scaffold, wherein each microfiber is aligned along and/or in parallel to a longitudinal axis, and wherein the layers are stacked one on top of the other; and a plurality of spacers, wherein each spacer is disposed between consecutive layers, thereby spacing therebetween.

According to some embodiments, the method further comprises (b) seeding and culturing a population of muscle cells on and/or within the at least one 3D scaffold of step (a), thereby enabling the formation of a 3D multi-layer muscle fibers structure thereon. According to some embodiments, the method further comprises (c) applying at least one dynamic mechanical loading stimulation to the at least one 3D scaffold comprising muscle fibers, of step (b), thereby affecting (e.g., enhancing) the production and/or secretion of extracellular vesicles (EVs) from the 3D multi-layer structure of muscle cells cultured thereon into a medium.

According to some embodiments, each spacer within the scaffold comprises a plurality of elongated spacing members, such that consecutive layers are spaced by the plurality of spacing members, wherein the spacing members are spaced apart in parallel from each other between consecutive layers, and wherein each spacing member is extending along a direction perpendicular to the longitudinal axis.

According to some embodiments, the plurality of spacing members within the scaffold comprise a first plurality and a second plurality, such that consecutive layers are spaced by said first and second pluralities of spacing members, wherein the first plurality of the parallel spacing members are disposed in the vicinity of the first end of the scaffold, wherein the second plurality thereof are disposed in the vicinity of the second end of the scaffold, and wherein the first and second pluralities of spacing members define an empty space therebetween.

According to some embodiments, a length of the empty space is greater than a distance between each two consecutive spacing members of any of the first plurality and any of the second plurality of spacing members. According to further embodiments, the length of the empty space is at least 10 times greater than the distance between each two consecutive spacing members of any of the first plurality and any of the second plurality of spacing members. According to some embodiments, the length of the empty space is greater than about 25% of a length of the 3D scaffold.

According to some embodiments, the scaffold comprises 2-20 layers of elastic microfibers, wherein each layer comprises 2-30 of parallel elastic microfibers.

According to some embodiments, each microfiber comprises one or more synthetic or natural polymers, selected from polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, and copolymers thereof. According to further embodiments, each microfiber comprises PDMS.

According to some embodiments, each microfiber has a diameter selected from the range of about 10 μm to about 1000 μm.

According to some embodiments, each spacing member is a microfiber having a diameter selected from the range of about 10 μm to about 1000 μm. According to some embodiments, each spacing member comprises one or more synthetic or natural polymers, selected from polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, and copolymers thereof.

According to some embodiments, the scaffold is configured to be stretched and to undergo more than about 10% strain without reaching the yield point thereof. According to some embodiments, the scaffold is configured to undergo more than about 20% strain without reaching the yield point thereof. According to some embodiments, the scaffold is configured to undergo more than about 25% strain without reaching the yield point thereof. According to some embodiments, the scaffold is configured to undergo more than about 100% strain without reaching the yield point thereof. According to some embodiments, the scaffold has a Young's modulus selected from the range of 0.1 to about 2 MPa.

According to some embodiments, step (a) further comprises providing a bioreactor system comprising at least one main chamber and a medium disposed therein, wherein the method comprises inserting the at least one 3D scaffold into the main chamber prior to step (c).

According to some embodiments, step (b) is performed within the main chamber.

According to some embodiments, the at least one main chamber accommodates therein at least two opposing platforms, wherein inserting the 3D scaffold into the main chamber comprises coupling said two opposing platforms to opposing portions of the 3D scaffold, such that the 3D scaffold is extending therebetween.

According to some embodiments, step (c) comprises displacing at least one of the two opposing platforms within the main chamber, thereby inducing mechanical loading stimulations on the elastic 3D scaffold extending therebetween, wherein the mechanical loading stimulations are selected from the group consisting of compression, tension (stretching), torsion, bending, and combinations thereof.

According to some embodiments, the two opposing platforms are coupled to opposing portions of the 3D scaffold, such that the plurality of microfibers are aligned along and/or in parallel to the longitudinal axis, wherein step (c) comprises displacing the two opposing platforms away from each other along the longitudinal axis, thereby inducing tension to the 3D scaffold extending therebetween.

According to some embodiments, step (c) comprises displacing the two opposing platforms away and towards each other repeatedly, thereby inducing repeating tension cycles to the 3D scaffold, at a certain frequency, for a certain time duration. According to some embodiments, the certain frequency is selected from the range of about 0.1 to about 5 Hz. According to further embodiments, the certain frequency is selected from the range of about 0.5 to about 5 Hz. According to some embodiments, the certain time duration is selected from the range of about 12 hours to about 21 days. According to further embodiments, the certain time duration is selected from the range of about 24 hours to about 7 days.

According to some embodiments, the extracellular vesicles produced by the methods of the present invention are selected from the group consisting of: exosomes, microvesicles, apoptotic bodies, ectosomes, and combinations thereof. According to further embodiments, the extracellular vesicles are exosomes.

According to some embodiments, the muscle cells are mammalian muscle cells. According to some embodiments, the mammalian muscle cells are human muscle cells. According to some embodiments, the human muscle cells are selected from: human skeletal muscle cells (SkMCs), human induced pluripotent stem cells derived-cardiomyocytes, human smooth muscle cells, and a combination thereof.

According to some embodiments, the method further comprises step (d) of collecting the medium of step (c). According to some embodiments, the method further comprises step (e) of isolating the secreted extracellular vesicles dispersed within the medium of step (d).

According to another aspect, there are provided extracellular vesicles produced according to the methods disclosed herein. According to some embodiments, the extracellular vesicles express at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof. According to further embodiments, the extracellular vesicles express at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein the expression of the at least one protein is upregulated compared to EVs produced without being subjected to at least one mechanical loading stimulation (i.e., under static/un-stretched conditions). According to yet further embodiments, the extracellular vesicles express a plurality of proteins selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein the expression of at least one of the proteins is upregulated compared to EVs produced without being subjected to at least one mechanical loading stimulation (i.e., under static/un-stretched conditions). According to some specific embodiments, the expression of plurality of the proteins is upregulated compared to EVs produced by static/un-stretched conditions.

According to another aspect, EVs secreted from muscle cells are provided, wherein the EVs are characterized by expressing at least one marker selected from CD9, CD63, and CD81, and expressing, in an upregulated amount compared to EVs produced without being subjected to at least one mechanical loading stimulation (i.e., under static/un-stretched conditions), at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof.

According to some embodiments, the EVs are characterized by expressing the markers CD9, CD63, and CD81, and expressing a plurality of proteins selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein at least one of the proteins is expressed in an upregulated amount compared to EVs produced by static/un-stretched conditions.

According to another aspect, there is provided a composition comprising a population of extracellular vesicles disclosed herein.

According to some embodiments, the extracellular vesicles or the composition as disclosed herein, are for use in the prevention or treatment of a disease or disorder. According to some embodiments, the disease or disorder is selected from the group consisting of: blood vessel diseases, cardiac diseases, skeletal muscle diseases, and combinations thereof.

According to another aspect, there is provided a method of prevention or treatment of a disease or disorder, comprising administering to a subject in need thereof a composition as disclosed herein above. According to some embodiments, the disease or disorder is selected from the group consisting of blood vessel diseases, cardiac diseases, skeletal muscle diseases, and combinations thereof.

According to another aspect, there is provided a three-dimensional (3D) scaffold configured to support a population of muscle cells seeded and cultured thereon, the scaffold comprising a plurality of layers, wherein each layer comprises a plurality of elastic microfibers spaced apart from each other, wherein each microfiber extends from a first end of the scaffold towards a second end of the scaffold, wherein each microfiber is aligned along and/or in parallel to a longitudinal axis, and wherein the layers are stacked one on top of the other; and a plurality of spacers, wherein each spacer is disposed between consecutive layers, thereby spacing therebetween, wherein the elastic 3D scaffold is configured to support an expansion of the population of muscle cells into a 3D multi-layer structure of muscle fibers.

According to some embodiments, each spacer within the scaffold comprises a plurality of elongated spacing members, such that consecutive layers are spaced by the plurality of spacing members, wherein the spacing members are spaced apart in parallel from each other between consecutive layers, and wherein each spacing member is extending along a direction perpendicular to the longitudinal axis.

According to some embodiments, the plurality of spacing members within the scaffold comprise a first plurality and a second plurality, such that consecutive layers are spaced by said first and second pluralities of spacing members, wherein the first plurality of the parallel spacing members are disposed in the vicinity of the first end of the scaffold, wherein the second plurality thereof are disposed in the vicinity of the second end of the scaffold, and wherein the first and second pluralities of spacing members define an empty space therebetween.

According to some embodiments, a length of the empty space is greater than a distance between each two consecutive spacing members of any of the first plurality and any of the second plurality of spacing members. According to further embodiments, the length of the empty space is at least 10 times greater than the distance between each two consecutive spacing members of any of the first plurality and any of the second plurality of spacing members. According to some embodiments, the length of the empty space is greater than about 25% of a length of the 3D scaffold.

According to some embodiments, the scaffold comprises 2-20 layers of elastic microfibers, wherein each layer comprises 2-30 of parallel elastic microfibers.

According to some embodiments, each microfiber comprises one or more synthetic or natural polymers, selected from polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, and copolymers thereof. According to further embodiments, each microfiber comprises PDMS.

According to some embodiments, each microfiber has a diameter selected from the range of about 10 μm to about 1000 μm.

According to some embodiments, each spacing member is a microfiber having a diameter selected from the range of about 10 μm to about 1000 μm. According to some embodiments, each spacing member comprises one or more synthetic or natural polymers, selected from polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, and copolymers thereof.

According to some embodiments, the scaffold is configured to be stretched and to undergo more than about 10% strain without reaching the yield point thereof. According to some embodiments, the scaffold is configured to undergo more than about 20% strain without reaching the yield point thereof. According to some embodiments, the scaffold has a Young's modulus selected from the range of 0.1 to about 2 MPa.

According to some embodiments, the scaffold is for use in producing extracellular vesicles (EVs) from muscle cells seeded and cultured thereon.

Certain embodiments of the present invention may include some, all, or none of the above advantages. Further advantages may be readily apparent to those skilled in the art from the figures, descriptions, and claims included herein. Aspects and embodiments of the invention are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following embodiments and aspects thereof are described and illustrated in conjunction with systems, tools and methods which are meant to be exemplary and illustrative, but not limiting in scope. In various embodiments, one or more of the above-described problems have been reduced or eliminated, while other embodiments are directed to other advantages or improvements.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

FIG. 1A illustrate a view in perspective of a scaffold 100, according to some embodiments.

FIG. 1B illustrate a cross sectional view in perspective of the scaffold 100 of FIG. 1A, according to some embodiments.

FIG. 1C illustrate a side view of the scaffold 100 of FIG. 1A, according to some embodiments.

FIG. 1D illustrate the scaffold 100 of FIG. 1A disposed within a main chamber 130, according to some embodiments.

FIG. 2 illustrate a flowchart of a method 200 for producing extracellular vesicles from muscle cells, according to some embodiments.

FIGS. 3A and 3B are photos of an elastic 3D scaffold with a defined fiber orientation, according to some embodiments.

FIG. 4A show a Boss Instron system used for measuring mechanical properties of the scaffold, according to some embodiments.

FIG. 4B show strain-stress curves of the elastic 3D scaffold utilizing the Instron system of FIG. 4A, according to some embodiments.

FIGS. 5A-5E represents Desmin and Dapi staining of SkMCs-seeded scaffolds under different magnifications: control scaffolds (FIGS. 5A-5C); and under cyclic stretch conditions (FIGS. 5D-5F).

FIGS. 6A and 6B represents EVs concentration (FIG. 6A) and size (FIG. 6B) analysis for both the stretch and control samples.

FIGS. 7A-7C represents Yap staining (light grey) for the control samples (FIG. 7A); stretched samples (FIG. 7B); and quantification of nucleus level (FIG. 7C). Data is presented as means±SEM (*<0.05).

FIGS. 8A-8C represents EVs flow cytometry graphs as detected using MACSplex exosome Kit on stretch induced EVs, labeled for the following markers: CD9 (FIG. 8A); CD63 (FIG. 8B), and CD81 (FIG. 8C).

FIG. 9 represents proteomic analysis of stretch-stimulated EVs compared with static EVs via a Volcano plot showing upregulated (left) and downregulated (right) proteins in flow-stimulated EVs compared with 3D static EVs.

### DETAILED DESCRIPTION OF THE INVENTION

The present invention provides systems and methods for inducing advanced production and/or secretion of extracellular vesicles from muscle cells.

As used herein, the terms “extracellular vesicles” and “EVs” are interchangeable, and refers to lipid bilayer-delimited particles that are released from cells naturally or following stimulations. The stimulations can include various dynamic mechanical loading profiles, such as cyclic compression, tension (stretching), torsion, bending, and combinations thereof.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure. In the figures, like reference numerals refer to like parts throughout.

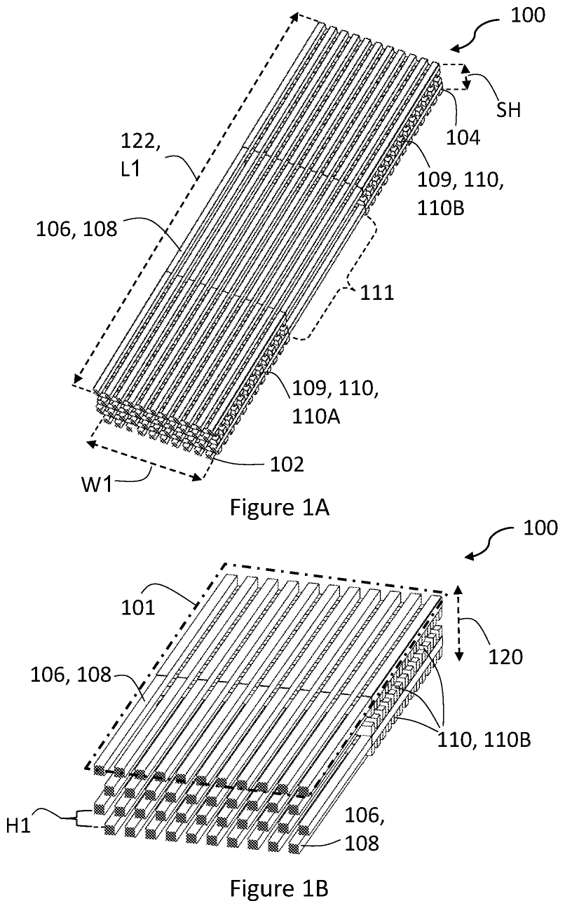

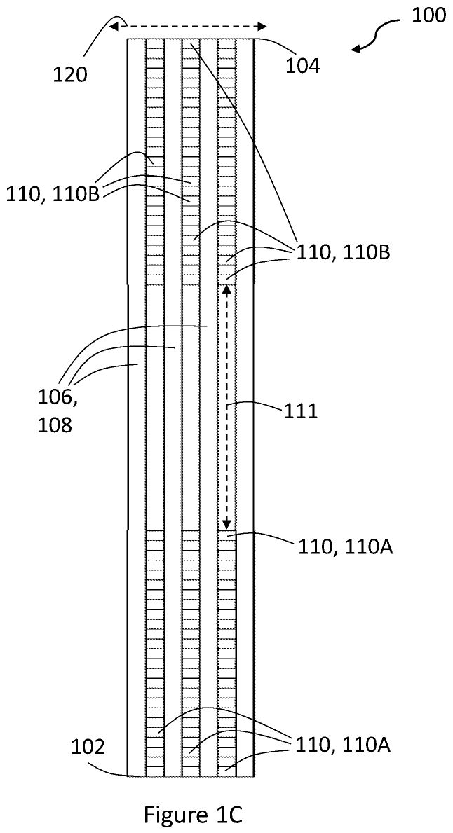

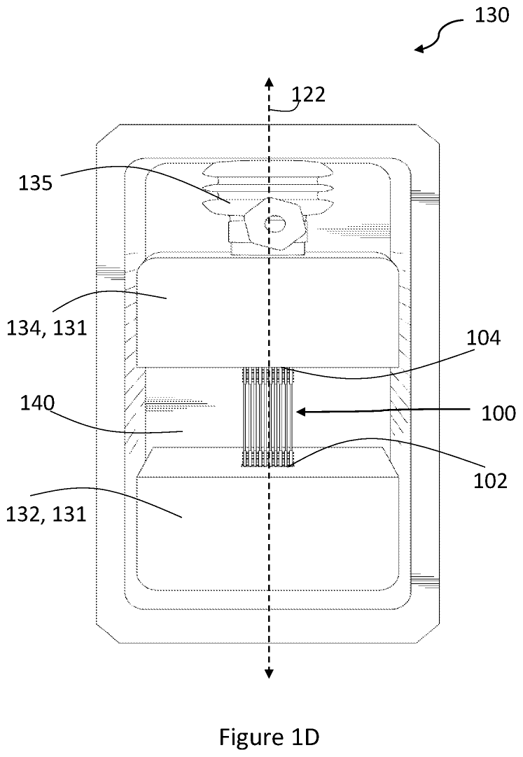

Reference is now made to FIGS. 1A-1D. FIG. 1A illustrate a view in perspective of a scaffold 100, according to some embodiments. FIG. 1B illustrate a cross sectional view in perspective of the scaffold 100 of FIG. 1A, according to some embodiments. FIG. 1C illustrate a side view of the scaffold 100 of FIG. 1A, according to some embodiments. FIG. 1D illustrate the scaffold 100 of FIG. 1A disposed within a main chamber 130, according to some embodiments.

According to a certain aspect, there is provided a system configured to induce or apply one or more dynamic mechanical loading profile(s) on at least one three-dimensional (3D) scaffold 100 and to a population of muscle cells cultured thereon, wherein said scaffold is appropriate for supporting seeding, growth and expansion of the cells and secretion of EVs from said cells. It is known that cells strongly respond to mechanical stimuli and adapt their behavior to loading conditions applied thereon. The development of organs and tissues like muscle fibers, bones, blood vessels and others, can be influenced by mechanical loading applied under various profiles (i.e., different types of loading profiles). The system of the present invention was specifically adapted to simulate in-vivo conditions suitable for muscle tissues, by applying direct loading on the culture substrate (i.e., scaffold 100), which was found critical to obtain tissues or cell structures with desired properties.

According to some embodiments, the system comprises at least one main chamber 130, as illustrated at FIG. 1D. According to further embodiments, the at least one main chamber 130 comprises a medium 140 disposed therein.

According to some embodiments, the main chamber 130 is a reactor. According to some embodiments, the main chamber 130 is a bioreactor. According to further embodiments, the system is a bioreactor system. According to some embodiments, the main chamber has a three-dimensional (3D) structure. According to some embodiments, the main chamber has a shape or a structure adapted to accommodate within at least one three-dimensional (3D) scaffold. According to some embodiments, the at least one 3D scaffold 100 is disposed within the main chamber 130.

According to some embodiments, the main chamber 130 is selected from the group consisting of: laminar flow reactor (LFR), plug flow reactor (PFR), continuous stirred-tank reactor (CSTR), batch reactor, heterogenous catalytic reactor, fed-batch bioreactor, perfusion bioreactor, fix-bed bioreactor, packed bed bioreactor, wave bioreactor, air lift bioreactor, vibrating bed bioreactor, and other known reactors or bioreactors in the art. Each possibility represents a separate embodiment.

According to some embodiments, the main chamber 130 is a bioreactor configured to enable to culture cells therein under mechanical loading. According to some embodiments, the main chamber 130 is a bioreactor configured to provide axial tension and/or compression loading onto at least one scaffold 100 disposed therein and to a population of muscle cells seeded and cultured thereon. According to some embodiments, the main chamber 130 is an Ebers TC3 bioreactor.

According to some embodiments, the main chamber 130 is configured to receive and/or to contain a medium 140 therein. According to some embodiments, the medium 140 is a growth medium or a culture medium, configured to support the growth of cells and microorganisms, such as muscle cells. According to some embodiments, the medium 140 comprises an aqueous solution. According to some embodiments, the medium 140 comprises at least one material selected from the group consisting of: water, salts, nutrients, minerals, vitamins, amino acids, nucleic acids, proteins (such as cytokines and growth factors), hormones, serum or any combination thereof. Each possibility represents a different embodiment.

According to some embodiments, the at least one 3D scaffold 100 comprises a population of muscle cells adhered thereto. According to some embodiments, the at least one 3D scaffold comprises a population of muscle cells seeded and cultured thereon. According to some embodiments, the population of muscle cells produced and/or secrets extracellular vesicles (EVs) into the medium disposed within the main chamber.

According to some embodiments, the muscle cells are stem cells derived muscle cells. According to some embodiments, the muscle cells are mammalian muscle cells. According to some embodiments, the muscle cells are human muscle cells. According to further embodiments, the muscle cells are selected from: human skeletal muscle cells (SkMCs), human induced pluripotent stem cells derived-cardiomyocytes, human smooth muscle cells, and a combination thereof. Each possibility represents a separate embodiment.

According to some embodiments, the extracellular vesicles secreted from the muscle cells are selected from the group consisting of: exosomes, microvesicles, apoptotic bodies and ectosomes. Each possibility represents a separate embodiment of the present invention.

As used herein, the term “exosomes” refers to membrane bound extracellular vesicles (EVs) that are produced in the endosomal compartment of most eukaryotic cells and later secreted from the cells. The exosomes typically contain various molecular components from the cells (also denoted “cargo” or “exosomal cargo”), that might include some or all of: proteins, lipids, mitochondrial components and genetic materials such as: RNA and DNA, and combinations thereof. According to some embodiments, the exosomal cargo comprises at least one protein. According to some embodiments, the exosomal cargo comprises at least one phospholipid or protein. According to some embodiments, the phospholipid is a membrane phospholipid. According to some embodiments, the protein is a membrane-based protein or a lipoprotein.

According to some embodiments, the extracellular vesicles (EVs) are exosomes. According to some embodiments, the extracellular vesicles are exosomes secreted from a population of muscle cells selected from human skeletal muscle cells (SkMCs), human induced pluripotent stem cells derived-cardiomyocytes, or both.

As used herein, the term “scaffold” refers to a three-dimensional structure comprising a material that provides a surface suitable for adherence/attachment and proliferation of cells. A scaffold may further provide mechanical stability and support. A scaffold may be in a particular shape or form so as to influence or delimit a three-dimensional shape or form assumed by a population of proliferating cells. According to some embodiments of the present invention, the scaffold is a three-dimensional substrate made from a material approved by a health authority, for human use.

According to some embodiments, there is provided an at least one 3D scaffold 100, configured to support a population of muscle cells seeded and/or cultured thereon, wherein said scaffold 100 is appropriate for supporting seeding, growth and expansion of the cells and secretion of EVs from said cells. According to some embodiments, the at least one 3D scaffold 100 comprises a population of muscle cells adhered thereto. According to some embodiments, the at least one 3D scaffold 100 comprises a population of muscle cells cultured thereon. According to some embodiments, the population of muscle cells produced and/or secrets extracellular vesicles (EVs) therefrom and into the medium disposed within the main chamber.

According to some embodiments, the at least one 3D scaffold 100 is disposed within a main chamber 130 and is configured to endure various dynamic mechanical loading stimulations therein. According to some embodiments, the at least one 3D scaffold 100 is immersed within the medium 140, which is disposed within the main chamber 130.

According to some embodiments, the at least one 3D scaffold 100 has a shape selected from the group consisting of a disc, a cube, hyperrectangle (a box), a cylinder, a sphere, or any other suitable polyhedron in the art. Each possibility represents a separate embodiment of the present invention.

According to some embodiments, the at least one 3D scaffold 100 comprises a plurality of parallel fibers. In further embodiments, each fiber is a microfiber 106. According to some embodiments, the at least one 3D scaffold 100 comprises a plurality of microfibers 106, wherein each microfiber 106 comprises at least one material selected from, but not limited to, (i) natural polymers or fibers selected from cellulose, silk, alginate, fibrin (fibrinogen), gelatin, collagen, hyaluronic acid (HA), chitosan, dextran sulfate, heparin, heparan sulfate, and functionalized derivatives thereof; and (ii) synthetic polymers selected from a polyester and a polyamide, such as polyacrylic acid derivatives and polyvinyl alcohol, including polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), as well as combinations thereof, that produce hydrogel polymer fibers useful in the invention. Each possibility represents a separate embodiment of the present invention.

According to some embodiments, each microfiber 106 comprises a polyolefin selected from but not limited to, polypropylene (PP), polyethylene (PE), and copolymers thereof. Each possibility represents a separate embodiment of the present invention. According to some embodiments, each microfiber 106 comprises one or more synthetic or natural polymers, selected from but not limited to, polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, and copolymers thereof. Each possibility represents a separate embodiment of the present invention. According to some embodiments, each microfiber 106 comprises polydimethylsiloxane (PDMS).

According to some embodiments, any one of the microfibers 106 can be extruded, printed, molded, leached, electro spun, or manufactured in any other suitable method in the art. Each possibility represents a separate embodiment of the present invention. According to further embodiments, the 3D scaffold 100 is manufactured by 3D printing. According to further embodiments, the plurality of microfibers 106 are manufactured by 3D printing.

According to some embodiments, the at least one 3D scaffold 100 is made from one or more biocompatible material(s). According to further embodiments, any one of the plurality of microfibers 106 is biocompatible. The term “biocompatible” as used herein, refers to materials having affinity with living tissues, low toxicity and no unacceptable foreign body reactions in the living body.

According to some embodiments, the at least one 3D scaffold 100 is elastic. According to some embodiments, the at least one elastic 3D scaffold 100 comprises a plurality of elastic microfibers 106, wherein each microfiber 106 extends from a first end 102 of the scaffold 100 towards a second end 104 of the scaffold 100, and is made from the material(s) as disclosed herein above. According to some embodiments, each microfiber 106 is aligned along and/or in parallel to a longitudinal axis 122, and to other microfibers 106 within the 3D scaffold 100.

As used herein, the terms “first end” and “second end” of the 3D scaffold 100 refers to opposite edges or ends thereof. It should be understood that if the scaffold 100 is in a 3D curvilinear shape such as an ellipsoid or a sphere, the terms “first end” and “second end” will refer to opposite edges or external surfaces thereof.

According to some embodiments, the at least one elastic 3D scaffold 100 comprises at least one layer 108 comprising one or more parallel microfibers 106. According to some embodiments, the at least one elastic 3D scaffold 100 comprises a plurality of layers 108, wherein each layer 108 comprises a plurality of parallel microfibers 106 aligned in the same direction and spaced apart from each other, i.e. in parallel and/or along to the longitudinal axis 122, as illustrated at FIG. 1A.

According to some embodiments, the at least one elastic 3D scaffold 100 further comprises a plurality of spacers 109, wherein each spacer 109 is disposed between consecutive layers 108, thereby defining a first height H1 spacing therebetween (see FIG. 1B). According to some embodiments, the plurality of layers 108 are vertically stacked, one on top of the other, in parallel to a vertical axis 120, wherein each layer 108 is spaced from the following layer 108 by each spacer 109. According to some embodiments, each layer 108 and each spacer 109 are disposed alternately one over the other, so that the first height H1 is formed between each two consecutive layers 108.

Advantageously, it is contemplated that spacing consecutive layers 108 by each spacer 109 defining the first height H1 therebetween, can enable the medium 140 to flow/enter into the scaffold 100 between consecutive layers 108, thereby allowing the medium to effectively support the growth and/or expansion of cells and microorganisms, such as the muscle cells, cultured on each of the microfibers 106.

According to some embodiments, each spacer 109 comprises a plurality of spacing members 110, such that consecutive layers 108 are spaced by the plurality of spacing members 110. According to some embodiments, the spacing members 110 are spaced apart in parallel from each other between consecutive layers 108. According to some embodiments, each spacing member 110 is extending along a direction perpendicular to the longitudinal axis 122, between consecutive layers 108. According to some embodiments, each spacing member 110 is extending along a direction perpendicular to the vertical axis 120 (see FIG. 1C).

According to some embodiments, the at least one elastic 3D scaffold 100 comprises a plurality of spacing members 110 configured to provide support to the plurality of layers 108 and maintain the first height H1 spacing between subsequent or consecutive layers 108 within the scaffold 100. According to some embodiments, each spacing member 110 is disposed vertically to the longitudinal axis 122 in the vicinity of at least one of the scaffold ends (i.e., end 102 and/or 104), between subsequent or consecutive layers 108, thereby providing support thereto and maintaining the first height H1 spacing between each two consecutive layers 108. According to some embodiments, each spacing member 110 is coupled or attached to the consecutive layers 108 it separates.

According to some embodiments, each spacing member 110 is elongated, that is the long dimension thereof (e.g., length) is greater than the short dimension (e.g., width or diameter) thereof. For example, the length of said elongated spacing member 110 may be at least three times, at least five times, at least ten times, or more, greater than of the width or diameter thereof. According to some embodiments, each spacing member 110 is a fiber. According to further embodiments, each spacing member 110 is a microfiber. According to some embodiments, each spacing member 110 is made of the same materials and/or has the same properties and/or diameter as each microfiber 106, as disclosed herein.

According to some embodiments, the plurality of spacing members 110 comprises a first plurality 110A and a second plurality 110B, such that consecutive layers 108 are spaced by said first and second pluralities 110A and 110B, respectively, of spacing members 110. According to some embodiments, the first plurality 110A of the parallel spacing members 110 are disposed in the vicinity of the first end 102 of the scaffold 100. According to some embodiments, the second plurality 110B thereof are disposed in the vicinity of the second end 104 of the scaffold 100, as illustrated at FIG. 1C.

As used herein, the term “vicinity” refers to a distance within a radius of less than about 8 mm of a given three-dimensional (3D) space. According to some embodiments, the term “vicinity” refers to a distance within a radius of less than about 5 mm, preferably less than about 3 mm, more preferably less than about 1 mm, or even more preferably less than about 0.5 mm of a given 3D space. Each possibility represents a separate embodiment of the present invention.

According to some embodiments, the first plurality 110A and the second plurality 110B of spacing members 110 define an empty space 111 therebetween (see FIG. 1C).

According to some embodiments, the length of the empty space 111 is greater than the distance between each two consecutive spacing members 110 of any of the first plurality 110A and any of the second plurality 110B of spacing members 110. According to some embodiments, the scaffold 100 comprises a plurality of empty spaces 111, wherein each empty space 111 is formed between each two consecutive layers 108 and between inner ends of the first plurality 110A and the second plurality 110B of spacing members 110 within the scaffold 100.

According to some embodiments, the length of the empty space 111, in parallel and/or along the longitudinal axis 122, is at least 5 times greater than the distance between each two consecutive spacing members 110 of any of the first plurality 110A and any of the second plurality 110B of spacing members 110. In further embodiments, the length of the empty space 111 is at least 10 times, optionally at least 15 times, alternatively at least 20 times, or more, greater than the distance between each two consecutive spacing members 110 of any of the first plurality 110A and any of the second plurality 110B thereof. Advantageously, the empty space 111 as disclosed herein above, may enable the medium to flow/enter into the scaffold 100 between consecutive layers 108, in order to effectively support the growth and/or expansion of cells and microorganisms thereon and therein.

Alternatively or additionally, in some embodiments, the at least one elastic 3D scaffold 100 is disposed within (or is enveloped by) a support structure configured to support and maintain the first height H1 spacing between consecutive layers 108, such as a frame or a surrounding housing (not shown). Said support structure may contain the plurality of spacing members 110 as disclosed above, optionally in addition to the other supporting element(s). Alternatively, the support structure does not contain the plurality of spacing members 110.

According to some embodiments, the elastic 3D scaffold 100 can comprise 2-20 layers 108 of elastic microfibers 106. According to some embodiments, the at least one elastic 3D scaffold 100 comprises at least two, at least three, at least four, at least five, or at least six layers 108 of elastic microfibers 106. Each possibility represents a different embodiment. According to some embodiments, the at least one elastic 3D scaffold 100 comprises at least four layers 108 of elastic microfibers 106, as illustrated at FIG. 1B. According to some embodiments, the at least one elastic 3D scaffold 100 comprises at least five layers 108 of elastic microfibers 106 (not shown).

According to some embodiments, each layer 108 of scaffold 100 comprises 2-30 of parallel elastic microfibers 106. According to further embodiments, each layer 108 of scaffold 100 comprises 5-15 of parallel elastic microfibers 106. According to still further embodiments, each layer 108 of scaffold 100 comprises 10 parallel elastic microfibers 106 (not shown). According to yet still further embodiments, each layer 108 of scaffold 100 comprises 11 parallel elastic microfibers 106, as illustrated at FIG. 1B.

According to some embodiments, the at least one elastic 3D scaffold 100 comprises a plurality of elastic microfibers groups (not shown). According to further embodiments, each group comprises a plurality of layers 108 which are vertically stacked one on top of the other in parallel to the vertical axis 120, wherein each layer 108 comprises a plurality of elastic microfibers 106 aligned in the same direction in parallel to the longitudinal axis 122, wherein each layer is spaced from the following layer by the first height H1. According to still further embodiments, the groups are vertically stacked one on top of the other along the vertical axis 120 and are spaced from each other by a second height H2 (not shown). The second height H2 can enable the medium to flow/enter into the scaffold and contact each cluster and thereby each layer 108.

According to some embodiments, the at least one elastic 3D scaffold 100 (and/or each microfiber 106) extends from the first end 102 towards the second end 104, defining a length L1 extending therebetween, in parallel to the longitudinal axis 122. According to some embodiments, the at least one elastic 3D scaffold 100 has a length L1 selected from a range of about 0.1 mm to about 2,500 mm. According to some embodiments, the length L1 of the at least one elastic 3D scaffold is above about 1 mm, alternately above about 5 mm, or optionally above about 10 mm. According to some embodiments, the length L1 of the at least one elastic 3D scaffold is selected from the range of: about 1 mm to about 100 mm, about 100 mm to about 200 mm, about 200 mm to about 500 mm, about 500 mm to about 1,000 mm, about 1,000 mm to about 1,500 mm, or about 1,500 mm to about 2,000 mm. Each possibility represents a different embodiment. According to some embodiments, the length L1 of the at least one elastic 3D scaffold is selected from the range of about 2 mm to about 50 mm. According to further embodiments, the length L1 of the at least one elastic 3D scaffold is selected from the range of about 5 mm to about 15 mm. According to a certain embodiment, the length L1 of the at least one elastic 3D scaffold is about 10 mm.

According to some embodiments, the at least one elastic 3D scaffold 100 has a width W1 selected from a range of about 0.1 mm to about 2,000 mm, wherein width W1 is vertical to the longitudinal axis 122. According to some embodiments, the width W1 is above about 1 mm or optionally above about 5 mm. According to some embodiments, the width W1 is selected from the range of about 1 mm to about 100 mm, about 100 mm to about 200 mm, about 200 mm to about 500 mm, about 500 mm to about 1,000 mm, about 1,000 mm to about 1,500 mm, or about 1,500 mm to about 2,000 mm. Each possibility represents a different embodiment. According to some embodiments, the width W1 is selected from the range of about 2 mm to about 50 mm. According to further embodiments, the width W1 is selected from the range of about 2 mm to about 15 mm. According to still further embodiments, the width W1 is selected from the range of about 3 mm to about 8 mm. According to a certain embodiment, the width W1 is about 5 mm.

According to embodiments, the width W1 and the length L1 of the scaffold 100 defines a plane 101 therebetween (see FIG. 1B). In some embodiments, the plane 101 is an XY plane. In some embodiments, each layer 108 of the scaffold 100 comprises a plurality of parallel microfibers 106 spaced apart from each other, wherein all parallel microfibers 106 substantially reside in the same plane 101.

As used herein, the term “substantially” refers to the complete or nearly complete extent or degree of an action, characteristic, property, state, structure, item, or result. For example, objects that are “substantially” in the same plane would mean that the objects are either completely reside in the same plane, or nearly completely reside in the same plane and may have a slight deviation therefrom. The exact allowable degree of deviation from plane completeness may in some cases depend on the specific context.

According to some embodiments, the at least one elastic 3D scaffold 100 has a scaffold height SH, optionally selected from a range of about 0.1 mm to about 300 mm, wherein the scaffold height SH is vertical to the plane 101, and wherein the scaffold height SH is parallel to the vertical axis 120. According to some embodiments, the scaffold height SH is above about 0.5 mm, alternately above about 1 mm, or optionally above about 1.5 mm. According to some embodiments, the scaffold height SH is selected from the range of about 0.1 mm to about 1 mm, about 1 mm to about 10 mm, about 10 mm to about 30 mm, about 30 mm to about 50 mm, about 50 mm to about 80 mm, or about 80 mm to about 100 mm. Each possibility represents a different embodiment. According to some embodiments, the scaffold height SH is selected from the range of about 0.5 mm to about 10 mm. According to further embodiments, the scaffold height SH is selected from the range of about 1 mm to about 3 mm. According to a certain embodiment, the scaffold height SH is about 1.6 mm.

According to some embodiments, the length of the empty space 111 in parallel and/or along the longitudinal axis 122 is greater than about 25% of the length L1 of the 3D scaffold 100. In further embodiments, the length of the empty space 111 is greater than about 30%, above about 50%, above about 70%, above about 80%, above about 90%, above about 95%, or more, of the length L1 of the 3D scaffold 100. Each possibility represents a different embodiment. According to some embodiments, the length of the empty space 111 is selected from the range of about 0.1 mm to about 2000 mm. In further embodiments, the length of the empty space 111 is selected from the range of about 2 mm to about 15 mm.

According to some embodiments, the at least one elastic 3D scaffold 100 is configured to undergo various profiles of cyclic mechanical loading stimulations. According to further embodiments, the at least one elastic 3D scaffold 100 is configured to return to its original 3D shape, following the termination of the mechanical loading applied thereon, due to its elastic qualities and properties.

According to some embodiments, the at least one elastic 3D scaffold 100 is stretchable. According to some embodiments, the scaffold 100 has a resilient 3D structure, wherein said resilient 3D structure enables the scaffold to be reversibly stretched and/or compressed. The resilient 3D structure of the scaffold 100 is formed to resiliently maintain its shape when not subjected to physical pressure (e.g., mechanical loading). The term “resilient”, as used herein with respect to the scaffold of the present invention (i.e., elastic 3D scaffold 100), refers to a scaffold being resistant to permanent deformation when such external force is applied thereto, and having a tendency to return to an original state/shape thereof, when the external force is no longer applied thereto.

According to some embodiments, the at least one elastic 3D scaffold 100 is configured to be cyclically stretched in opposite directions, along or in parallel to the longitudinal axis 122, and to undergo more than about 1% strain without reaching the scaffold's yield point, or without suffering from any type of permanent deformation or failure. Strain represents the displacement between particles (i.e., elongation) of the scaffold 100 during the application of tensile stress (i.e., axially stretching), relative to a reference length (i.e., length L1). According to some embodiments, the at least one elastic 3D scaffold 100 is configured to undergo more than about 5%, 10%, 20%, 30%, 40%, 50%, 60%, 70%, 80%, 90% or 100% strain without reaching the scaffold's yield point. Each possibly represents a different embodiment. According to further embodiments, the at least one elastic 3D scaffold 100 is configured to undergo more than about 20% strain without reaching the scaffold's yield point. According to yet still further embodiments, the at least one elastic 3D scaffold 100 is configured to undergo more than about 25% strain without reaching the scaffold's yield point.

As used herein, the term “yield point” refers to the point on a stress-strain curve that indicates the limit of elastic behavior of a material and the beginning of plastic behavior thereof. Prior to the yield point, a material will deform elastically and will return to its original dimensions (i.e., shape) when the applied stress (i.e., force) is removed, and thus will exhibit resilient qualities. Once the applied stress is increased to a level at which the yield point is passed, at least a portion of the deformation caused to the material by the applied force will be permanent and non-reversible, and is known as plastic deformation. As used herein, the terms “Young's modulus” or “elastic modulus” are interchangeable, and refers the slope of the stress-strain curve (i.e., the ratio between tensile stress to tensile strain) in the elastic region thereof.

According to some embodiments, the at least one elastic 3D scaffold 100 has a Young's modulus in the range of 0.05 to about 2.5 MPa. According to further embodiments, the at least one elastic 3D scaffold 100 has a Young's modulus in the range of 0.1 to about 1 MPa. According to still further embodiments, the at least one elastic 3D scaffold 100 has a Young's modulus in the range of 0.2 to about 1 MPa. According to certain embodiments, the at least one elastic 3D scaffold 100 has a Young's modulus in the range of 0.2 to about 0.5 MPa.

According to some embodiments, each microfiber 106 has a diameter in the range of about 1 μm to about 1000 μm. According to further embodiments, the diameter of the microfibers 106 is in the range of about 50 μm to about 500 μm. According to still further embodiments, the diameter of the microfibers 106 is in the range of about 100 μm to about 300 μm. According to yet still further embodiments, the diameter of the microfibers 106 is in the range of about 150 μm to about 250 μm. According to certain embodiments, the diameter of the microfibers 106 is about 200 μm.

According to some embodiments, each spacing member 110 has a diameter in the range of about 1 μm to about 1000 μm. According to further embodiments, the diameter of each spacing member 110 is in the range of about 50 μm to about 500 μm. According to some embodiments, each spacing member 110 has a diameter which identical to the diameter of each microfiber 106.

According to some embodiments, the distance between each two consecutive spacing members 110, of any of the first plurality 110A and any of the second plurality 110B of spacing members 110, is below about 5%, alternatively below about 1%, optionally below about 0.1%, or less, of the length L1 of the 3D scaffold 100. According to some embodiments, the distance between each two consecutive spacing members 110, of any of the first plurality 110A and any of the second plurality 110B of spacing members 110, is about the size of the diameter of each spacing member 110 (or less). According to some embodiments, the distance between each two consecutive spacing members 110, of any of the first plurality 110A and any of the second plurality 110B of spacing members 110, is selected from about 50 μm to about 500 μm. In further such embodiments, the distance between each two consecutive spacing members 110, of any of the first plurality 110A and any of the second plurality 110B of spacing members 110, is less than about 300 μm.

According to some embodiments, the first height H1 is selected from the range of about 0.1 μm to about 1000 μm. According to further embodiments, the first height H1 is in the range of about 50 μm to about 500 μm. According to still further embodiments, the first height H1 is in the range of about 100 μm to about 300 μm. According to yet still further embodiments, the first height H1 is in the range of about 150 μm to about 250 μm. According to certain embodiments, the first height H1 is about 200 μm. According to some embodiments, the first height H1 is identical to the diameter of each microfiber 106 and/or the diameter of each spacing member 110.

According to some embodiments, the second height H2 is selected from the range of about 100 μm to about 5 mm. According to further embodiments, the second height H2 is in the range of about 500 μm to about 2 mm. According to still further embodiments, the second height H2 is in the range of about 700 μm to about 1.5 mm. According to still further embodiments, the second height H2 is about 1 mm.

It is contemplated, in some embodiments, that the unique structural design and dimensions of the elastic 3D scaffold 100 as disclosed herein above, facilitates the formation of a multi-layer expansion in the form of a three-dimensional (3D) multi-layer structure of muscle fibers (i.e., mayofibers), wherein the muscle cells adhere to the scaffold 100 and/or to each other to form connected muscle multi-layer mayofibers having a favorable defined orientation (i.e., in parallel to the longitudinal axis 122), after differentiation and maturation on the 3D scaffold. As was disclosed herein above, the unique design of the scaffold 100 comprises a plurality of layers 108, wherein each layer comprises a plurality of parallel microfibers 106 aligned in the same direction in parallel to the longitudinal axis 122, and wherein consecutive layers 108 are spaced by a plurality of spacing members 110.

According to some embodiments, the at least one main chamber 130 is adapted to accommodate therein the at least one elastic 3D scaffold 100 having the 3D multi-layer structure of muscle fibers adhered thereto, and to subject the at least one elastic 3D scaffold 100 to various dynamic mechanical loading profiles, wherein the muscle fibers secrete extracellular vesicles into the medium 140 disposed within the main chamber. According to further embodiments, the mechanical loading profiles are selected from, but not limited to, compression, tension (stretching), torsion, bending, and combinations thereof. Each possibly represents a different embodiment.

As used herein, the terms “mechanical loading profiles” and “mechanical loading stimulations” are interchangeable, and refers to inducing or generating repeating cycles of various mechanical stimuli on the at least one elastic 3D scaffold 100 disposed within the at least one main chamber. Each cycle comprises applying force on the scaffold 100 and then relaxing the applied force thereon, thereby allowing the scaffold to return to its original shape. The repeating cycles are generated at a certain frequency, for a certain time duration.

According to some embodiments, the at least one main chamber 130 comprises one or more inner movable member(s) disposed therein configured to be coupled to one or more portion(s) of the elastic 3D scaffold 100, thus enabling to induce or generate cyclic mechanical loading stimulations thereon. According to some embodiments, the at least one main chamber 130 accommodate therein at least one platform 131, configured to be coupled to at least a portion of the elastic 3D scaffold 100. According to some embodiments, the at least one main chamber accommodate therein at least one platform 131, configured to be coupled to or to support at least a portion of the first end 102 of the scaffold 100, the second end 104 thereof, or both. According to further embodiments, the at least one main chamber accommodate therein at least two opposing platforms 131, wherein each platform is configured to be attached/coupled to at least a portion of each one of the first and second scaffolds ends 102 and 104, repressively.

According to some embodiments, the at least two opposing platforms 131 of the main chamber 130 comprises a first platform 132 and a second platform 134, as illustrated at FIG. 1D. According to some embodiments, the first platform 132 and the second platform 134 are coupled to different opposing portions of the scaffold 100, such that the scaffold 100 is extending therebetween. According to some embodiments, the first platform 132 is coupled to at least a portion of the scaffold 100 residing in the vicinity of the first end 102 thereof. In further such embodiments, the first platform 132 is coupled to a portion of the scaffold 100 comprising the first plurality 110A of spacing members 110. According to some embodiments, the second platform 134 is coupled to at least a portion of the scaffold 100 residing in the vicinity of the second end 104 thereof. According to further such embodiments, the second platform 134 is coupled to a portion of the scaffold 100 comprising the second plurality 110B of spacing members 110.

According to some embodiments, each one of the two opposing platforms 131 (i.e., the first and second platforms, 132 and 134, respectively) comprises coupling means configured to grip the scaffold 100 or a portion thereof, when coupled thereto. The coupling means may be clamps, wherein the clamps are adapted to enable the coupling of each scaffold portion to each respective platform.

According to some embodiments, at least one of the two opposing platforms 131 (i.e., the first and second platforms, 132 and 134, respectively) is configured to be movable or displaced within the main chamber 130. In some embodiments, the first platform 132 is static and the second platform 134 is movable (illustrated at FIG. 1D). In further embodiments, the second platform 134 is coupled to at least one actuator 135 configured to enable the displacement or movement thereof within the main chamber 130. In alternative embodiments, both the first and second platforms 132 and 134, respectively, are movable (not shown).

According to some embodiments, the two opposing platforms 131 of the main chamber 130 are configured to induce cyclic mechanical loading profiles at a certain frequency for a certain time duration, on the scaffold 100 extending therebetween and coupled thereto. According to some embodiments, the certain frequency is selected from the range of about 0.1 to about 5 Hz. According to further embodiments, the certain frequency is selected from the range of about 0.5 to about 4 Hz. According to further embodiments, the certain frequency is selected from the range of about 0.5 to about 1.5 Hz. According to still further embodiments, the certain frequency is about 1 Hz. According to some embodiments, the certain time duration is selected from the range of about 2 hours to about 30 days. According to further embodiments, the certain time duration is selected from the range of about 2 hours to about 21 days. According to further embodiments, the certain time duration is selected from the range of about 6 hours to about 10 days. According to further embodiments, the certain time duration is selected from the range of about 12 hours to about 10 days. According to still further embodiments, the certain time duration is selected from the range of about 1 day to about 5 days. According to still further embodiments, the certain time duration is about 2 days.

According to some embodiments, the first platform 132 and the second platform 134 of the main chamber 130 are coupled to opposite portions of the scaffold 100 as disclosed herein above, and are configured to induce or generate repeating compression cycles on the elastic 3D scaffold 100 extending therebetween. According to further embodiments, the first platform 132 and the second platform 134 are configured to be cyclically advanced towards each other along or in parallel to the longitudinal axis 122, in order to compress the elastic 3D scaffold 100 extending therebetween, and then to be distanced from each other in order to allow the elastic 3D scaffold 100 to relax, and optionally to return to its original shape.

According to some embodiments, the first platform 132 and the second platform 134 of the main chamber 130 are coupled to opposite portions of the scaffold 100 as disclosed herein above, and are configured to induce repeating torsion cycles on the elastic 3D scaffold 100 extending therebetween. According to further embodiments, the first platform 132 and the second platform 134 are configured to be cyclically rotated/displaced at opposing directions relative to each other, in order to apply torsion to the elastic 3D scaffold 100 extending therebetween, and then to be rotated back to their original configuration or positions, in order to allow the elastic 3D scaffold 100 to relax, and optionally to return to its original shape.

According to some embodiments, the first platform 132 and the second platform 134 of the main chamber 130 are coupled to opposite portions of the scaffold 100 as disclosed herein above, and are configured to induce repeating bending cycles on the elastic 3D scaffold 100 extending therebetween. According to further embodiments, the first platform 132 and the second platform 134 are configured to be cyclically displaced relative to each other, in order to bend the elastic 3D scaffold 100 extending therebetween, and then displaced back to their original configuration or positions in order to allow the elastic 3D scaffold 100 to relax, and optionally to return to its original shape. According to some embodiments, the at least one main chamber accommodating within the at least two opposing platforms further comprise a mechanical and/or electrical member configured to facilitate the cyclic bending of the elastic 3D scaffold 100.

According to some embodiments, the first platform 132 and the second platform 134 of the main chamber 130 are coupled to opposite portions of the scaffold 100 as disclosed herein above, and are configured to induce or generate repeating tension cycles (i.e., stretching) to the elastic 3D scaffold 100 extending therebetween. The tension loading (i.e., stretching) is generated along the same direction/axis as the specific fiber alignment within the scaffold 100, meaning that the scaffold 100 is being stretched in opposite directions along and/or in parallel to longitudinal axis 122, wherein the plurality of parallel microfibers 106 are aligned along and/or in parallel to longitudinal axis 122, as illustrated at FIG. 1D.

According to some embodiments, the first platform 132 and the second platform 134 are configured to be axially displaced (i.e. distanced) from each other along the longitudinal axis 122, in order to stretch (provide tension) the elastic 3D scaffold 100 extending therebetween, and then to be advanced or brought back towards one another in order to allow the elastic 3D scaffold 100 to relax, and optionally to return to its original shape.

As used herein, the term “repeating tension cycles” refers to applying repeating cycles of stretching and relaxing the scaffold 100, wherein the stretching is performed by pulling/displacing the scaffold in opposite directions.

It is contemplated, in some embodiments, that in order to mimic the 3D dynamic environment which is physiologically applied on various muscle tissues within the human body, cyclic axial tension should be induced on the at least one elastic 3D scaffold 100 in the same orientation/direction of which the parallel microfibers 106 are aligned relative to each other. Advantageously, providing repeating tension cycles in parallel to the longitudinal axis 122 onto the elastic 3D scaffold 100 enables to induce physiological changes in the population of muscle cells cultured thereon, resulting in enhanced production and/or secretion of extracellular vesicles therefrom, and in some embodiments result in improved biological effect of the extracellular vesicles on muscle cells. As was disclosed above, the scaffold 100 comprises the plurality of layers 108 comprising the plurality of parallel microfibers 106 aligned in parallel to the longitudinal axis 122, so that the repeating tension cycles are being provided to the scaffold 100 along the same axis as the plurality of microfibers 106 are aligned (i.e., longitudinal axis 122).

As used herein, the term “defined orientation” refers to the alignment of the plurality of microfibers 106 of the scaffold 100 in parallel to the longitudinal axis 122 and to each other.

According to some embodiments, the at least one main chamber 130 is adapted to accommodate therein a plurality of elastic 3D scaffolds 100. According to some embodiments, the system of the present invention further comprises at least one additional main chamber 130 adapted to accommodate therein at least one additional elastic 3D scaffold 100, and to enable inducing or generating various dynamic mechanical loading profiles thereon. According to further embodiments, the system further comprises a plurality of additional main chambers 130.

According to some embodiments, the main chamber 130 is adapted to accommodate therein the at least one elastic 3D scaffold 100 and the medium 140 as disclosed herein above, wherein the medium is static, and wherein the medium does not flow through or within the main chamber.

According to alternative embodiments, the main chamber 130 is adapted to accommodate therein the at least one elastic 3D scaffold 100, and to enable perfusion medium flow through the main chamber. As used herein, the terms “perfusion medium flow” or “perfusion flow” are interchangeable, and refers to a perfusion main chamber, wherein the medium enters the perfusion main chamber and flows therethrough.

According to some embodiments, the main chamber 130 is a perfusion bioreactor. As used herein the terms “perfusion bioreactor” or “open loop perfusion configuration” are interchangeable, and refers to a bioreactor system which is able to continuously feed cells disposed and cultured therein with fresh media while remove spent media. Typically, the fresh media is provided to the cells at the same rate as the spent media is removed. By continuously removing spent media and replacing it with new media, nutrient levels within the perfusion bioreactor are maintained for optimal growing conditions, while cell waste products are removed in order to avoid toxicity.

According to some embodiments, the system as presented herein above is a bioreactor system, wherein the main chamber 130 is a perfusion bioreactor, and the bioreactor system further comprises a filtering apparatus, said filtering apparatus is configured to filter the spent media exiting main chamber, in order to separate waste products from secreted extracellular vesicles disposed within the medium. The waste products can be separated from the bioreactor system and be disposed of. The secreted extracellular vesicles can continue to circulate within the bioreactor system. Optionally, the secreted extracellular vesicles can be separated from bioreactor system, collected and maintained in an external apparatus or storing device.

According to some embodiments, the system further comprises at least one or more sensors for measuring in the medium at least one parameter selected from the group consisting of pressure, flow rate, temperature, pH, dissolved oxygen, concentration of medium components and extracellular vesicles quantity or concentration. Each possibility represents a separate embodiment of the present invention. According to some embodiments, the system further comprises one or more temperature-control elements for controlling the temperature within the main chamber.

The concentration of extracellular vesicles within the medium can be calculated by the number of extracellular vesicles divided by the volume of the medium within a defined space, such as the volume of the main chamber. The concentration of extracellular vesicles within the medium can be also calculated by the number of extracellular vesicles divided by the cross-sectional area of a defined space.

According to some embodiments, the system further comprises a control unit in operative communication with the at least one or more sensors, configured to receive measurements of the at least one parameter and adjust the at least one parameter based on the measurements. According to some embodiments, the control unit is further configured to control the movement of the at least two opposing platforms relative to one another, in order to induce the cyclic mechanical loading profiles on the elastic 3D scaffold 100 extending therebetween.

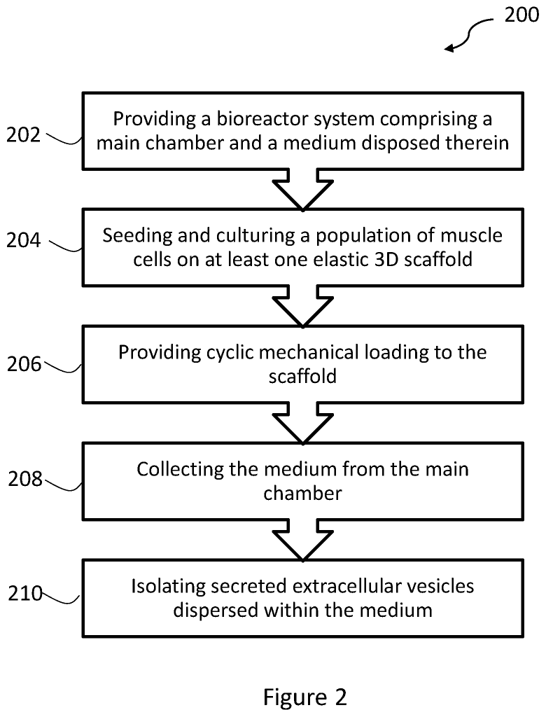

Reference is now made to FIG. 2, showing a flowchart of a method 200 for producing extracellular vesicles from muscle cells, according to some embodiments of the present invention.

According to another aspect, the present invention provides a method 200 for producing extracellular vesicles from muscle cells, the method comprising step 202 of providing the bioreactor system as disclosed herein above, wherein the bioreactor system comprises the main chamber 130 as disclosed herein above, wherein the main chamber 130 comprises the medium 140 disposed therein. According to some embodiments, the bioreactor system is configured to induce cyclic mechanical loading profiles on the at least one elastic 3D scaffold 100 and to a population of muscle cells cultured thereon, as disclosed herein above.

According to some embodiments, step 202 further comprises providing the at least one elastic 3D scaffold 100 as disclosed herein above, and optionally placing or inserting it within the main chamber 130.

According to some embodiments, placing or inserting the at least one elastic 3D scaffold 100 within the main chamber 130 comprises coupling a portion of the scaffold 100 to the first platform 132 and an opposite portion thereof to the second platform 134, so that the scaffold 100 extends between the two opposing platforms. In some preferred embodiments, the scaffold 100 is coupled to the first platform 132 and to the second platform 134 so that the plurality of parallel microfibers 106 of the scaffold 100 are aligned in parallel to the longitudinal axis 122 (see FIG. 1D).

According to some embodiments, the method further comprises step 204 of seeding and culturing a population of muscle cells on and/or within the at the least one elastic 3D scaffold 100. In some embodiments, the seeding and culturing is performed on at least a portion of the plurality of parallel microfibers 106 of the scaffold 100. In some embodiments, the seeding and culturing is performed on at least a portion of an external surface of each microfiber 106, optionally between consecutive layers 108. In some embodiments, the seeding and culturing is performed on the external surface of each microfiber 106, within the empty space 111, between consecutive layers 108.

According to some embodiments, step 204 of culturing a population of muscle cells on the at least one elastic 3D scaffold 100 is performed within the main chamber 130. According to other embodiments, step 204 of culturing a population of muscle cells on the at least one elastic 3D scaffold 100 is performed outside of the main chamber 130.

According to some embodiments, step 204 comprises: providing a certain amount of muscle cells, and seeding them on to the at least one elastic 3D scaffold 100, thereby adhering them thereto. According to some embodiments, the certain amount of muscle cells is in the range of about 0.001 to about 10 million muscle cells. According to further embodiments, the certain amount is in the range of about 0.1 to about 1.5 million muscle cells. According to still further embodiments, the certain amount is about 0.75 million muscle cells.

According to some embodiments, step 204 comprises providing conditions for the formation of a 3D multi-layer structure of muscle fibers cultured on and/or within the 3D scaffold 100. Said conditions can be provided to the scaffold 100 and to the cells cultured thereon within the main chamber 130, or outside thereof.

According to some embodiments, the conditions for the formation of a 3D multi-layer structure of muscle fibers comprises pre-coating the elastic 3D scaffold 100 with fibronectin for a time duration selected from about 0.1 hour to about 10 hours, at a temperature selected from the range of about 4-50° C., under various possible humidity values. According to further such embodiments, the conditions comprise pre-coating the elastic 3D scaffold 100 with fibronectin for a time duration selected from about 0.5 hour to about 3 hours, at a temperature of about 37° C., and a humidity of about 85-95%.

According to some embodiments, the conditions further comprise suspending the muscle cells in a solution comprising at least one of thrombin and fibrinogen. According to further embodiments, the conditions further comprise removing the muscle cells from the solution and immediately seeding them onto the scaffold 100, wherein the seeding comprise providing a temperature selected from the range of about 4-40° C., under various possible humidity values, for a time duration of about 5 minutes to 5 hours. According to further such embodiments, the seeding comprises providing a temperature of about 37° C. and a humidity of about 85-95%, for a time duration of about 15 minutes to 1.5 hours.

According to some embodiments, the conditions further comprise culturing or incubating the cells on the scaffolds at a temperature selected from the range of about 4-40° C. under various possible humidity values, first in a suitable growth medium for about 12 hours to about 10 days, and then in the medium as presented herein above for about 1 day to about 3 months. According to further such embodiments, the conditions further comprise culturing the cells on the scaffolds at a temperature of about 37° C. and a humidity of about 85-95% (and 5% CO₂), first in a suitable growth medium for about 1 day to about 5 days, and then in the medium as presented herein above for about 1 week to about 2 months.

According to some embodiments, the method further comprises step 206 of providing or generating various dynamic mechanical loading profiles to the at least one elastic 3D scaffold 100 and to the population of muscle cells cultured thereon, wherein the population of muscle cells secretes extracellular vesicles into the medium 140. It is contemplated, in some embodiments, that the dynamic mechanical loading profiles significantly affects (i.e., enhances) the production and/or secretion of extracellular vesicles from the 3D multi-layer structure of muscle cells into the medium.

According to some embodiments, if step 204 was performed outside of the main chamber, step 206 initially comprises placing or inserting the at least one elastic 3D scaffold 100 into the main chamber, as was disclosed herein above, prior to providing the dynamic mechanical loading stimulations thereto.

According to some embodiments, the at least one elastic 3D scaffold 100 is placed or inserted into the main chamber such that the plurality of parallel microfibers 106 are aligned along and/or in parallel to the same axis as the repeating tension cycles are being provided to (i.e., the longitudinal axis 122).

According to some embodiments, step 206 further comprises inducing or generating repeating tension (stretching) cycles on the elastic 3D scaffold, along and/or in parallel to the longitudinal axis 122. The tension (stretching) cycles include applying tensile stress (i.e., axially stretching) on the scaffold 100 along/in parallel to the longitudinal axis 122. According to some embodiments, step 206 comprises axially displacing (i.e., distancing) the first platform 132 and the second platform 134 away and towards each other repeatedly, along the longitudinal axis 122, in order to stretch (provide tension) the elastic 3D scaffold 100 extending therebetween, at a certain frequency, for a certain time duration.

According to some embodiments, the certain frequency is selected from the range of about 0.1 to about 5 Hz. According to further embodiments, the certain frequency is selected from the range of about 0.5 to about 5 Hz. According to some embodiments, the certain time duration is selected from the range of about 6 hours to about 21 days. According to further embodiments, the certain time duration is selected from the range of about 12 hours to about 5 days.

Since the previously known protocols for extracellular vesicles production are mainly based on 2D static cultivation of cells, they can only provide limited yields of extracellular vesicles production (Rome, Sophie, et al. “Skeletal muscle-released extracellular vesicles: State of the art.” Frontiers in physiology 10 (2019): 929). Advantageously, the present investors have discovered that by providing repeating tension cycles onto the at least one elastic 3D scaffold 100 as disclosed above, wherein the repeating tension cycles are being provided to the scaffold 100 along the same axis as the plurality of microfibers 106 are aligned (i.e., along and/or in parallel to the longitudinal axis 122), the extracellular vesicles production and/or secretion from the population of muscle cells seeded and cultured thereon into the medium can be significantly enhanced. Furthermore, the resulting secreted EVs may have improved or enhanced properties, relative to secreted EVs which were produced under different conditions.

According to some embodiments, the method further comprises step 208 of collecting the medium 140 from the main chamber 130.

According to some embodiments, the method further comprises step 210 of isolating the secreted extracellular vesicles dispersed within the medium 140. Any method known in the art for collecting and/or isolating EVs from a medium may be used according to the present invention. According to some embodiments, the method of isolating the EVs from the medium is selected from the group consisting of: Ultracentrifugation (UC), Density gradient UC, Ultrafiltration (UF), Tangential Flow Filtration (TFF), Hydrostatic dialysis, Precipitation kits/polymer (PEG or others), Size Exclusion Chromatography (SEC), Affinity Chromatography, Immuno-isolation (FACS, MACS), Microfluidic Devices, and combinations thereof. Each possibility represents a separate embodiment of the present invention.

According to some embodiments, the secreted extracellular vesicles are isolated utilizing a differential centrifugation procedure including Ultracentrifugation (UC).

According to some embodiments, the combination of the at least one elastic 3D scaffold 100 having the 3D multi-layer structure of muscle fibers adhered thereto, while inducing various dynamic mechanical loading profiles thereon as disclosed herein above, result not only in enhanced production of extracellular vesicles (preferably exosomes) therefrom, but also in morphological changes of the muscle fibers and in improved properties of the extracellular vesicles secreted, e.g. an improved pro-angiogenic effect.

EVs produced by the above methods and systems as well as compositions comprising at least one exosome produced by said methods and systems, are also within the scope of the present invention. According to some embodiments, the extracellular vesicles (EVs) comprise at least one component selected from the group consisting of: proteins, polypeptides, peptides, amino acids, lipids, mitochondrial components and polynucleotide sequences. According to some embodiments, the extracellular vesicles comprise a genetic material such as RNA and DNA. According to some embodiments, the extracellular vesicles comprise at least one engineered genetic material. According to some embodiments, the extracellular vesicles comprise at least one protein. According to some embodiments, the extracellular vesicles comprise at least one protein produced by muscle cells engineered to produce said protein. According to some embodiments, the extracellular vesicles comprise at least one phospholipid. According to some embodiments, the phospholipid is a membrane phospholipid. According to some embodiments, the protein is a membrane-based protein or a lipoprotein.

According to some embodiments, the extracellular vesicles produced by the above methods and systems express at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof. According to further embodiments, the extracellular vesicles express at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein the expression of the at least one protein is upregulated compared to EVs produced without being subjected to at least one mechanical loading stimulation (i.e., under static/un-stretched conditions). According to yet further embodiments, the extracellular vesicles express a plurality of proteins selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein the expression of at least one of the proteins is upregulated compared to EVs produced without being subjected to at least one mechanical loading stimulation (i.e., under static/un-stretched conditions). According to some specific embodiments, the expression of the plurality of the proteins produced by the above methods and systems is upregulated, compared to EVs produced without being subjected to mechanical loading stimulations (i.e., under static/un-stretched conditions).

According to some embodiments, EVs secreted from muscle cells are provided, wherein the EVs are characterized by expressing at least one marker selected from CD9, CD63, and CD81, and expressing, in an upregulated amount compared to EVs produced without being subjected to at least one mechanical loading stimulation (i.e., under static/un-stretched conditions), at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof. According to some embodiments, the EVs are characterized by expressing the markers CD9, CD63, and CD81, and expressing a plurality of proteins selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein at least one of the proteins is expressed in an upregulated amount compared to EVs produced by static/un-stretched conditions.

According to some embodiments, said EVs may be used for any application known in the art for exosomes, including but not limited to diagnostics, preventive and therapeutic applications such as tissue remodeling, tissue repair or tissue regeneration, neural disease treatment, diabetic and ischemic disease treatment, cardiovascular disease treatment, psychiatric disease treatment, vaccines, cancer treatment, immune disorders treatment, wound healing, and cosmetic applications. Each possibility represents a separate embodiment of the present invention.

As used herein, the terms “treating” and “treatment” refer to a method of alleviating or abrogating a disease and/or its attendant symptoms.

According to some embodiments, a composition comprising EVs produced by the above methods and systems is also within the scope of the present invention. According to some embodiments, a pharmaceutical composition or a cosmetic composition comprising said EVs produced by the above methods and systems is also within the scope of the present invention.

Any disease or disorder eligible for diagnostics, prevention or treatment with muscle cells may be treated or prevented with a composition comprising EVs produced by the above methods and systems, according to the present invention.

According to some embodiments, the EVs produced by the methods of the present invention may be used for prevention and treatment of a variety of diseases and disorders, and in particular muscle-related diseases and disorders. According to further embodiments, the muscle-related diseases and disorders are selected from the group consisting of blood vessel diseases, cardiac diseases, skeletal muscle diseases, and combinations thereof. Each possibility represents a separate embodiment of the present invention.

According to some embodiments, a disease or disorder eligible for prevention or treatment with compositions comprising EVs produced by the methods of the present invention is selected from the group consisting of: blood vessel diseases, cardiac diseases, skeletal muscle diseases, and combinations thereof. Each possibility represents a separate embodiment of the present invention.

Methods of preventing or treating a disease or disorder comprising administering a composition comprising EVs produced according to the present invention are also included.

The EVs of the present invention and the compositions comprising them, may be administered using any method known in the art, including but not limited to parenteral, enteral and topical routes.

The term “plurality”, as used herein, means more than one.

The term “about”, as used herein, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to the disclosed devices, systems and/or methods.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. No feature described in the context of an embodiment is to be considered an essential feature of that embodiment, unless explicitly specified as such.

Although the invention is described in conjunction with specific embodiments thereof, it is evident that numerous alternatives, modifications and variations that are apparent to those skilled in the art may exist. It is to be understood that the invention is not necessarily limited in its application to the details of construction and the arrangement of the components and/or methods set forth herein. Other embodiments may be practiced, and an embodiment may be carried out in various ways. Accordingly, the invention embraces all such alternatives, modifications and variations that fall within the scope of the appended claims.

### EXAMPLES

### Example 1—Scaffold Fabrication, 3D Cell Seeding and Proliferation

The following muscle cells were used in the examples: primary human skeletal muscle cells (SkMCs), purchased from PromoCell and cultured in the recommended PromoCell Skeletal Muscle Cell Growth Medium. The differentiation medium of the SkMCs was composed of Dulbecco's Modified Eagle Medium 1× (Gibco), 5% horse serum (Sigma Aldrich), 1% penicillin-streptomycin (Biological Industries), and 1% Glutamax (Gibco).

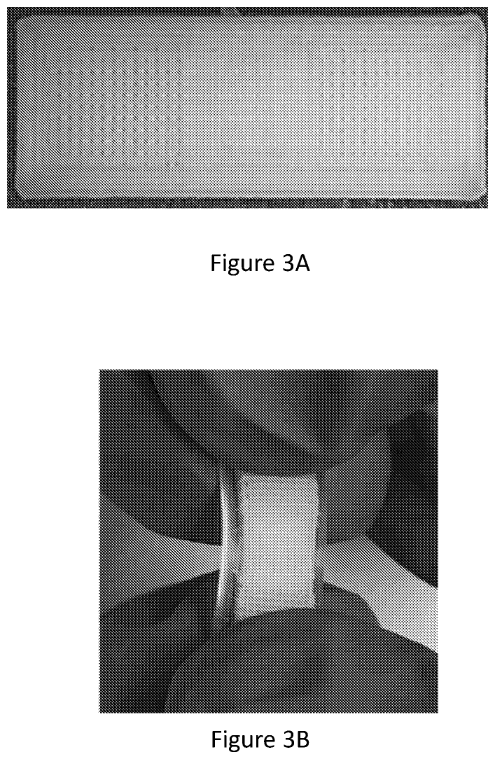

For culturing SkMCs in a 3D environment, elastic 3D supportive scaffolds comprising a plurality of layers, each layer comprises parallel microfibers having a defined orientation in parallel to each other were specifically fabricated, similarly to scaffold 100 as disclosed herein above. The fabrication method of the elastic 3D scaffolds relies on micro-resolution 3D printing of negative mold made of water-soluble sacrificial material (BVOH) followed by polymer casting and mold removal. Printed constructs were designed on SolidWorks and sliced in 3D Slicer (Prusa version). Using a 3D printer (Prusa), a Butenediol Vinyl Alcohol Co-polymer (BVOH) mold was printed, having dimensions of: a length of 10 mm, a width of 5 mm, and a height of 1.6 mm. Said mold contained aligned fibers (200 μm diameter). Polydimethylsiloxane (PDMS) polymers were prepared using Sylgard Silicone elastomer base (Downcorning, polymer) and Sylgard Silicone elastomer curing agent (Downcorning, crosslinker) at a ratio of 10:1. Afterwards, the PDMS polymers were casted into the printed mold, for the fabrication of the elastic 3D scaffolds. This step was followed by lyophilization overnight. The constructs were then dissolved in distilled water for 6 h and dried at room temperature. The resulting scaffolds are illustrated at FIGS. 3A and 3B.

It is contemplated that the unique design of the elastic 3D scaffolds as disclosed herein above can lead to the formation multi-layers of muscle fibers with defined orientation guided by the supportive PDMS elastic fibers, which can mimic the 3D dynamic environment which is physiologically applied on various muscle tissues.

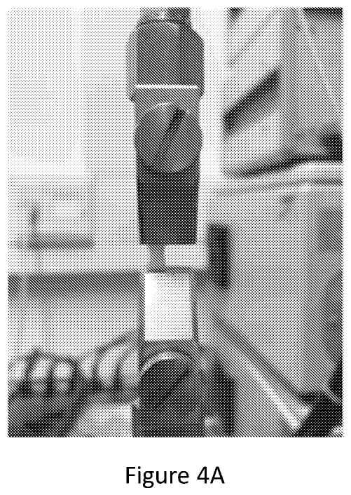

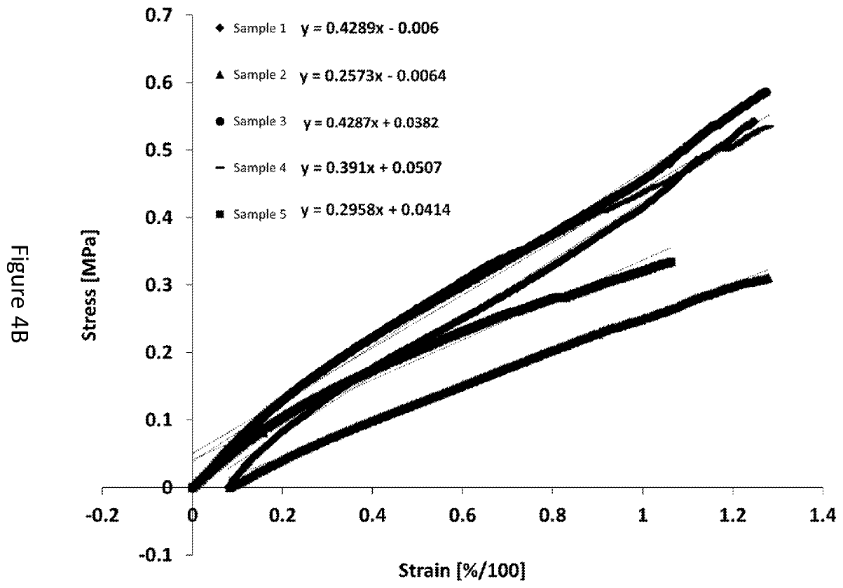

The mechanical properties of the PDMS elastic scaffold were assessed using Boss Instron system (FIG. 4A). Five scaffold samples were tested, revealing a typical Young's modulus (average Young's modulus of 0.36034 MPa) with excellent resilience. All samples stretched for more than 120% stain levels, without reaching the material yield point or observing any other failure, and all of the scaffold samples remained fully intact (FIG. 4B).

In order to perform optimal 3D cell seeding and to expand in multi-layers of muscle fibers, the elastic 3D scaffolds were pre-coated with 60 μl fibronectin (3%) for 1h, in an incubator. After trypsinization, 0.75*10⁶ SkMCs were suspended with 20 μl thrombin (50U) and 20 μl fibrinogen (50 mg/ml), and seeded immediately onto the scaffolds. After 30 min incubation, 2 ml growth medium was added to each scaffold in a 12-well plate. After 3 days culture in the growth medium, the scaffolds were supplemented with differentiation medium for 3 weeks.

### Example 2—Dynamic Mechanical Loading Experiment Protocol

After 3-week culture in the differentiation medium, the 3D elastic SkMCs-seeded scaffolds having multi-layered expansion of muscle fibers thereon were assembled into an Ebers TC3 bioreactor system for applying dynamic mechanical loading stimulations thereto, in the form of cyclic tension loading profile (i.e., stretching), in order to mechanically induce EVs secretion. The cyclic stretch (1 Hz, 25% strain) was applied for 2 days, i.e. the scaffolds samples underwent repeating tension (stretching) cycles resulting in 25% strain, in parallel to the direction of the PDMS fibers alignment within the scaffold, at a frequency of 1 Hz, for 2 days.

Control scaffolds samples were additionally tested: 3D static control scaffolds sample were similarly seeded and cultivated at static conditions, with no cyclic tension loading profile (i.e., stretch condition) applied thereto.

After stretching for 2 days in the bioreactor, EVs isolation was performed using a differential centrifugation protocol described in C. Thery, S. Amigorena, G. Raposo, and A. Clayton, “Isolation and characterization of EVs from cell culture supernatants and biological fluids.,” Curr. Protoc. cell Biol., vol. Chapter 3, p. Unit 3.22, April 2006. In brief, conditioned medium from the bioreactor was collected for a series of centrifugations (300 g for 10 min, 2,000 g for 10 min, 10,000 g for 30 min). The collected supernatant was ultracentrifuged at 100,000 g for 70 min. After washing the pellet with PBS, a second round of ultracentrifugation at 100,000 g for 70 min was performed. The final pellet was resuspended in 200 μl PBS. The EVs number was counted using a Nanosight NS500.

For immunofluorescence staining, the scaffolds were fixated in 4% PFA for 15 min, permeabilized with 0.3% Triton for 10 min, and then blocked in 5% BSA solution for 2 h. Next, the scaffolds were incubated with goat-anti-desmin antibody (sc-7559, 1:100) and mouse-anti-YAP (Santa cruz, 1:100) in 5% BSA, overnight, at 4° C. Scaffolds were then incubated with donkey-anti-goat 546 (1:800, Invitrogen) and donkey-anti-mouse-488 (1:400, Invitrogen) and DAPI (1:1000, Sigma), for 3 h at room temperature. Finally, scaffolds were imaged using confocal microscope (Zeiss LSM700).

### Example 3—Muscle Cell Morphology and Viability

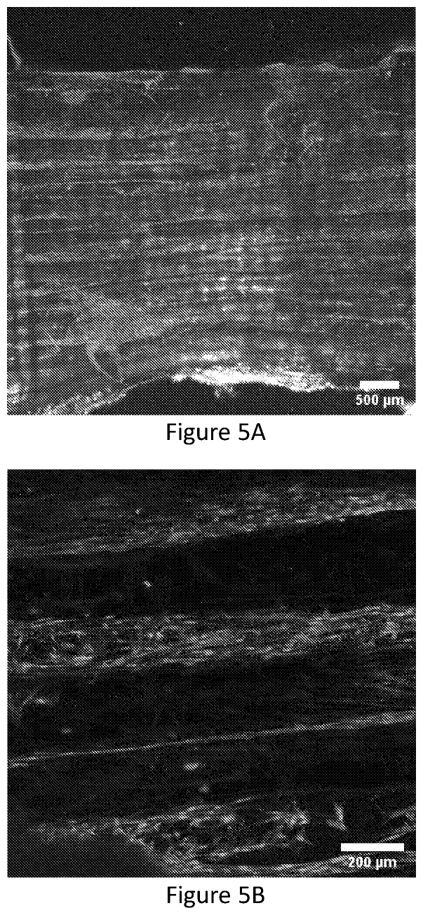

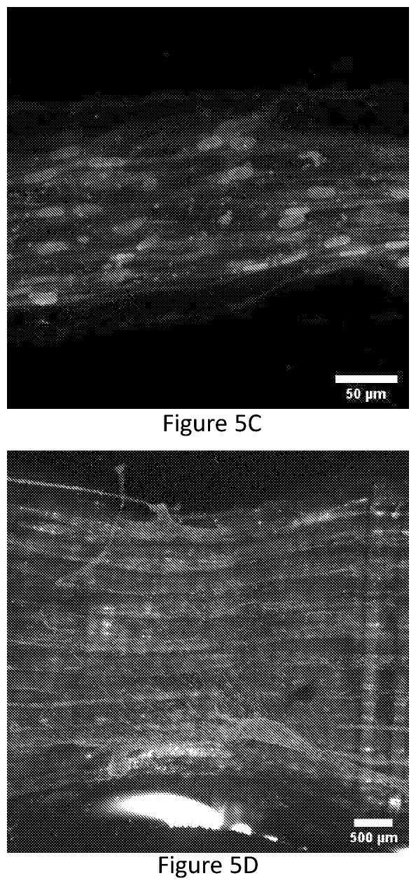

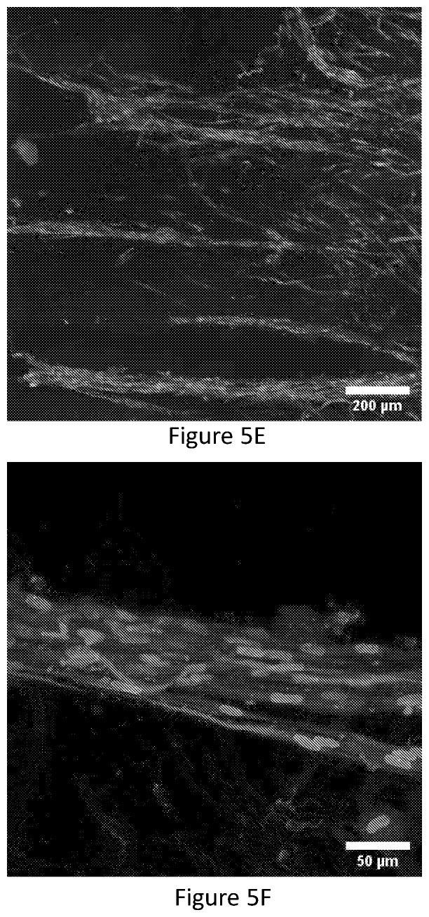

The impact of dynamic mechanical loading (i.e., stretching) on cell viability, morphology, maturity and muscle fibers defined orientation was assessed utilizing Desmin and Dapi staining of the SkMCs-seeded cyclic stretched scaffolds and the control unstretched scaffolds (FIGS. 5A-5F). Both cell nucleus (stained by DAPI) and cytoplasm (stained by Desmic) obtained an elongated and thin morphology, indicating on mature elongated multi-nuclei myotubes. This morphology change is a typical indication of the cells responding to the loading stimulations (FIGS. 5D-5F), as compared to the 3D static control (FIGS. 5A-5C).

It is contemplated that the morphology changes and muscle fiber maturation of the cells is a result of the unique design of the 3D supportive scaffold having the defined fiber orientation as was presented herein above, which guides the muscle fibers remodeling post-seeding, both in the stretched samples (FIGS. 5D-5F) and in the control samples (FIGS. 5A-5C).

### Example 4—EVs Characterization

The effect of the stretched scaffolds versus control unstretched scaffold samples on EVs production evaluated. The conditioned medium was collected from the bioreactors and the EVs were purified by ultra-centrifugation, followed by size and concentration analysis using the Nanosight system as explained at Example 2.

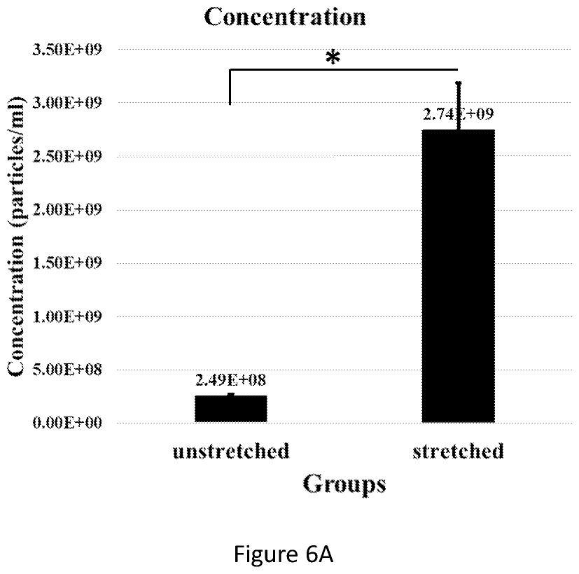

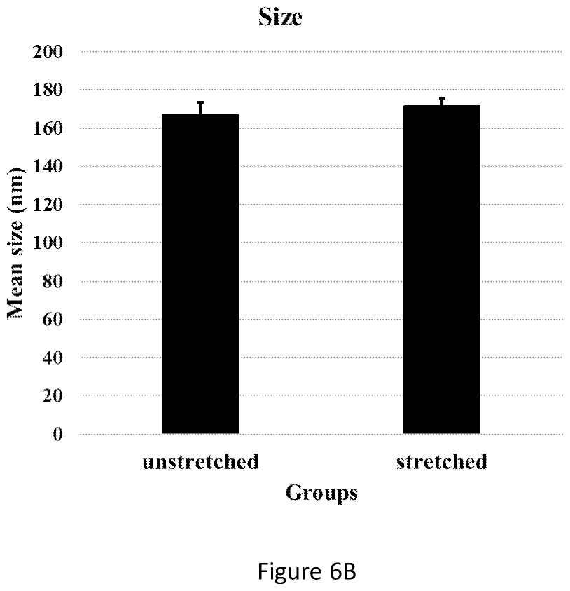

While the EVs mean size showed similar values for both stretched scaffold and control scaffold samples, the EVs concentration analysis revealed dramatic change reflected in an 11-fold higher EVs production level in the stretched scaffolds as compared to the control samples (FIGS. 6A and 6B).

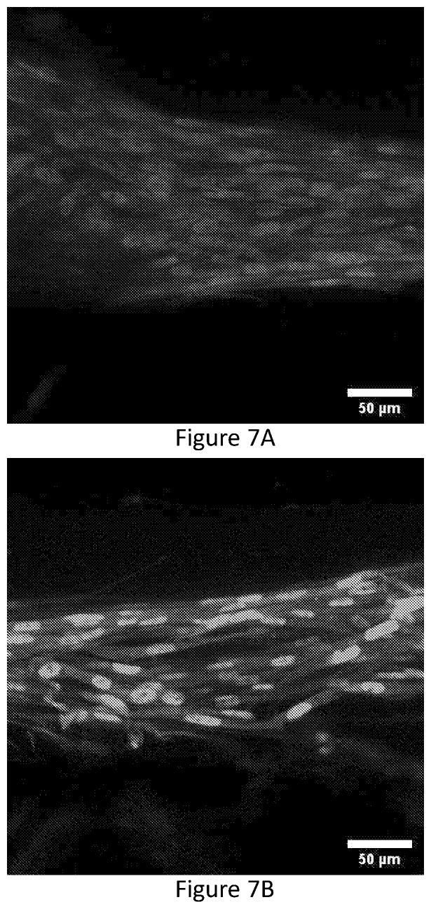

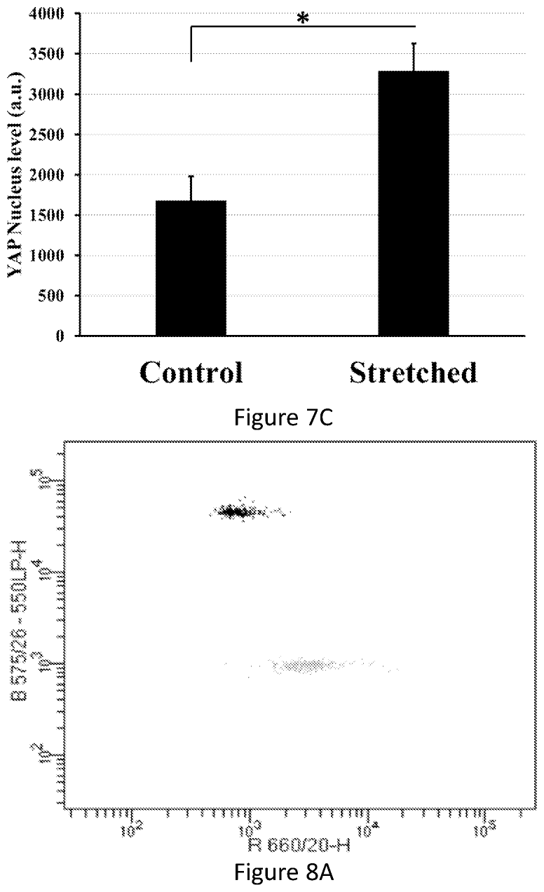

In addition, Yes-associated protein (YAP) staining was conducted was indication for cell mechano-sensing. Under mechanical loading YAP is translocated and over-expressed in the cell nucleus. The significant increase of YAP expression in the cell nuclei in the stretched samples confirmed that the cells experienced significant mechanical loading compared to the unstretched control samples (FIGS. 7A-7C).

### Example 5—Fluorescence-Activated Cell Sorting (FACS) of EVs

To characterize key EV markers, MACSplex exosome kit (Miltenyi Biotech) was used, as reported previously. Briefly, the overnight capture antibody incubation protocol was applied allowing detection of 37 exosomal surface markers. FACS analysis was carried out on the BD LSR-II Analyzer (BD Biosciences). For the analysis, the surface markers values were compared to the corresponding control antibody included in the kit and considered as the measurement threshold.

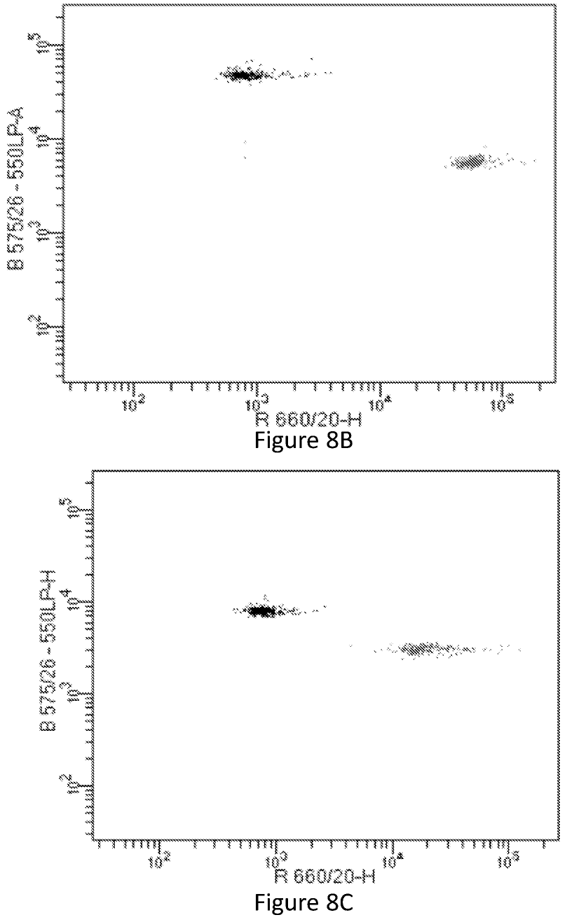

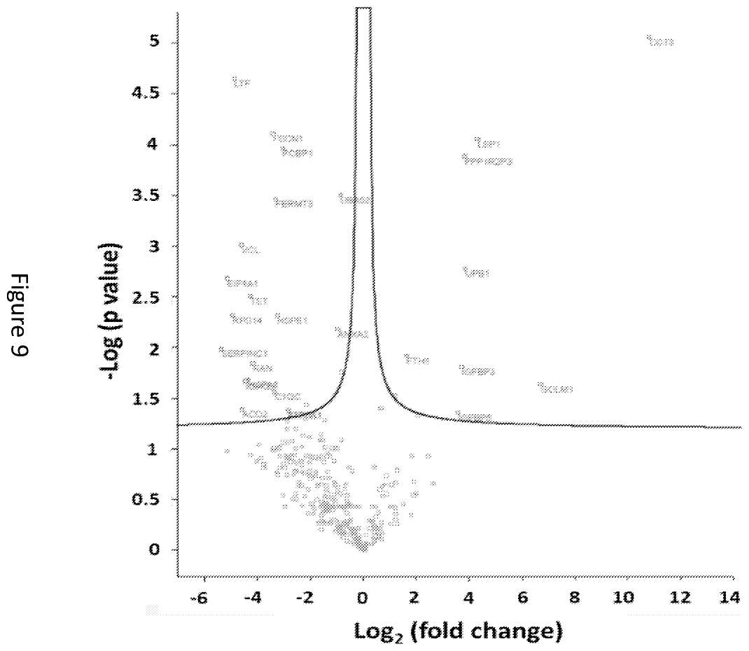

As can be seen at FIGS. 8A-8C, the key EV markers CD9, CD63, and CD81 were detected. The EV clusters shifted right in the graphs indicate the captured EVs for the specific markers as compared to control beads on the left.

### Example 6—Proteomics Characterization

EVs isolated from the control samples or stretch-stimulated samples (n=3/group) were digested with trypsin and the secreted peptides were analyzed by liquid chromatography-tandem mass spectrometry on a Q-Exactive plus (Thermo Fisher Scientific). Data was analyzed with MaxQuant software with false discovery rate (FDR)<0.01 and additional analysis was done in Perseus. Protein table was filtered to eliminate the identifications from the reverse database, and common contaminants were moved to another tab. The results also filtered out proteins that appeared in only one repeat. The intensities were transformed to log 2 and missing values were replaced by 18 in log 2 which was the baseline intensity. A t-test was performed to compare the intensities of the two groups. The differential proteins with Q value less than 0.05 and log 2 fold change above 1 (fold change 2) and at least 2 peptides, were labeled.

A Volcano plot was generated to indicate increased and decreased protein expressions in the flow-stimulated group (FIG. 9). From the proteomics analysis, 185 proteins identified, 160 were upregulated in the stretch-stimulated samples compared to samples produced by static static/un-stretched conditions (see Table 1), and 25 showed decreased expression (see Table 2). The upregulated proteins in bold in Table 1 are those associated with the production protocols of the present invention, e.g., mechanical transduction machinery, cell remodeling, etc.

TABLE 1
upregulated proteins

| | | | Intensity |
|---|---|---|---|
| | | | difference |
| | | | Log 2 Stretch |
| Protein IDs | Protein names | Gene names | vs. control |
| P01008 | Antithrombin-III | SERPINC1 | 5.375150681 |
| P60842; Q14240; | Eukaryotic initiation factor 4A-I; | EIF4A1; | 5.158092499 |
| P38919 | Eukaryotic initiation factor 4A-II | EIF4A2 | |
| P62263 | 40S ribosomal protein S14 | RPS14 | 4.937773387 |
| P02788 | Lactotransferrin; Lactoferricin-H; Kaliocin- | LTF | 4.878896713 |
| | 1; Lactoferroxin-A; Lactoferroxin- | | |
| | B; Lactoferroxin-C | | |
| P18206 | Vinculin | VCL | 4.62313652 |
| Q99798 | Aconitate hydratase, mitochondrial | ACO2 | 4.583912532 |
| P11142; P54652 | Heat shock cognate 71 kDa protein | HSPA8 | 4.360235214 |
| Q16762 | Thiosulfate sulfurtransferase | TST | 4.280021032 |
| Q8TCU6 | Phosphatidylinositol 3,4,5-trisphosphate- | PREX1 | 4.270457586 |
| | dependent Rac exchanger 1 protein | | |
| P62826 | GTP-binding nuclear protein Ran | RAN | 4.187068303 |
| P07858 | Cathepsin B; Cathepsin B light | CTSB | 4.033137004 |
| | chain; Cathepsin B heavy chain | | |
| P34931 | Heat shock 70 kDa protein 1-like | HSPAIL | 3.949407578 |
| P01009 | Alpha-1-antitrypsin; Short peptide from AAT | SERPINA1 | 3.877707163 |
| P12829 | Myosin light chain 4 | MYL4 | 3.796725591 |
| P62328 | Thymosin beta-4; Hematopoietic system | TMSB4X | 3.78848203 |
| | regulatory peptide | | |
| Q16658 | Fascin | FSCN1 | 3.426170985 |
| P35232 | Prohibitin | PHB | 3.406223297 |
| P09651; | Heterogeneous nuclear ribonucleoprotein | HNRNPA1 | 3.39609464 |
| A0A2R8Y4L2; | A1; Heterogeneous nuclear ribonucleoprotein | | |
| Q32P51 | A1, N-terminally processed | | |
| P02747 | Complement C1q subcomponent subunit C | CIQC | 3.335564931 |
| Q86UX7 | Fermitin family homolog 3 | FERMT3 | 3.322409312 |
| P0C0L4; P0C0L5 | Complement C4-A; Complement C4 beta | C4A; C4B | 3.292418162 |
| | chain; Complement C4-A alpha chain; C4a | | |
| | anaphylatoxin;C4b-A; C4d-A; Complement | | |
| | C4 gamma chain; Complement C4-B; | | |
| | Complement C4 beta chain; Complement | | |
| | C4-B alpha chain; C4a anaphylatoxin; C4b-B; | | |
| | C4d-B; Complement C4 gamma chain | | |
| Q99623 | Prohibitin-2 | PHB2 | 3.20007515 |
| P48047 | ATP synthase subunit O, mitochondrial | ATP5O | 3.165116628 |
| P19823 | Inter-alpha-trypsin inhibitor heavy chain H2 | ITIH2 | 3.149845759 |
| P29966 | Myristoylated alanine-rich C-kinase | MARCKS | 3.146430333 |
| | substrate | | |
| P01023 | Alpha-2-macroglobulin | A2M | 3.062946955 |
| Q15365 | Poly(rC)-binding protein 1 | PCBP1 | 3.057002385 |
| Q00325 | Phosphate carrier protein, mitochondrial | SLC25A3 | 3.011579514 |
| P14625 | Endoplasmin | HSP90B1 | 3.004646301 |
| P60174 | Triosephosphate isomerase | TPI1 | 2.999156952 |
| A0A075B6P5; | Ig kappa chain V-II region FR; Ig kappa | IGKV2D-28; | 2.959355036 |
| A0A087WW87; | chain V-II region Cum; Ig kappa chain V-II | IGKVA18; | |
| P01615; P01614; | region RPMI 6410 | IGKV2D-30; | |
| A2NJV5; | | IGKV2D-29 | |
| A0A0A0MRZ7; | | | |
| A0A075B6S6; | | | |
| A0A075B6S2; | | | |
| P06310 | | | |
| P60660; P14649 | Myosin light polypeptide 6; Myosin light | MYL6; MYL6B | 2.930739721 |
| | chain 6B | | |
| P61247 | 40S ribosomal protein S3a | RPS3A | 2.928260167 |
| P17066; P48741 | Heat shock 70 kDa protein 6; Putative heat | HSPA6; HSPA7 | 2.886822383 |
| | shock 70 kDa protein 7 | | |
| P02656 | Apolipoprotein C-III | APOC3 | 2.884578705 |
| P38646 | Stress-70 protein, mitochondrial | HSPA9 | 2.882835388 |
| Q8IW75 | Serpin A12 | SERPINA12 | 2.865541458 |
| P25705 | ATP synthase subunit alpha, mitochondrial | ATP5A1 | 2.838994344 |
| P80188 | Neutrophil gelatinase-associated lipocalin | LCN2 | 2.836078008 |
| P05023; P13637; | Sodium/potassium-transporting ATPase | ATP1A1; | 2.752423604 |
| P50993; Q13733; | subunit alpha-1; Sodium/potassium- | ATP1A3; | |
| P54707; P20648 | transporting ATPase subunit alpha- | ATP1A2 | |
| | 3; Sodium/potassium-transporting ATPase | | |
| | subunit alpha-2 | | |
| P27105 | Erythrocyte band 7 integral | STOM | 2.734057109 |
| | membrane protein | | |
| P32119 | Peroxiredoxin-2 | PRDX2 | 2.702742894 |
| P25788 | Proteasome subunit alpha type-3 | PSMA3 | 2.695151647 |
| P04004 | Vitronectin; Vitronectin V65 | VTN | 2.651873271 |
| | subunit; Vitronectin V10 | | |
| | subunit; Somatomedin-B | | |
| P21333 | Filamin-A | FLNA | 2.651367823 |
| P62491; Q15907 | Ras-related protein Rab-11A; Ras-related | RAB11A; | 2.604408264 |
| | protein Rab-11B | RAB11B | |
| P06733; P09104 | Alpha-enolase | ENO1 | 2.591204961 |
| P49411 | Elongation factor Tu, mitochondrial | TUFM | 2.556460063 |
| P35613 | Basigin | BSG | 2.538743973 |
| P08670; P17661; | Vimentin | VIM | 2.538267136 |
| P41219; Q16352; | | | |
| P07196; P07197 | | | |
| P00441 | Superoxide dismutase [Cu-Zn] | SOD1 | 2.528121948 |
| Q13423 | NAD(P) transhydrogenase, mitochondrial | NNT | 2.515751521 |
| P48735 | Isocitrate dehydrogenase [NADP], | IDH2 | 2.445885976 |
| | mitochondrial | | |
| P01876; P01877 | Ig alpha-1 chain C region | IGHA1 | 2.434834162 |
| P01857 | Ig gamma-1 chain C region | IGHG1 | 2.341340383 |
| P02647 | Apolipoprotein A-I; Proapolipoprotein A- | APOA1 | 2.331502914 |
| | I; Truncated apolipoprotein A-I | | |
| P08195 | 4F2 cell-surface antigen heavy chain | SLC3A2 | 2.327769597 |
| P54709 | Sodium/potassium-transporting ATPase | ATP1B3 | 2.321832657 |
| | subunit beta-3 | | |
| P12814; O43707 | Alpha-actinin-1; Alpha-actinin-4 | ACTN1; ACTN4 | 2.312669754 |
| P02545 | Prelamin-A/C; Lamin-A/C | LMNA | 2.303098043 |
| P07237 | Protein disulfide-isomerase | P4HB | 2.271755854 |
| P26641 | Elongation factor 1-gamma | EEF1G | 2.268718719 |
| P08238; Q58FF7 | Heat shock protein HSP 90-beta | HSP90AB1 | 2.218753815 |
| P08123 | Collagen alpha-2(I) chain | COL1A2 | 2.217879613 |
| P80723 | Brain acid soluble protein 1 | BASP1 | 2.157211304 |
| P68363; Q9BQE3; | Tubulin alpha-1B chain; Tubulin alpha-1C | TUBA1B; | 2.089126587 |
| Q71U36; P0DPH8; | chain; Tubulin alpha-1A chain; Tubulin | TUBA1C; | |
| P0DPH7; Q6PEY2; | alpha-3E chain | TUBA1A; | |
| Q9NY65; A6NHL2 | | TUBA3E | |
| Q14315 | Filamin-C | FLNC | 2.076173782 |
| P07437; Q9BVA1; | Tubulin beta chain; Tubulin beta-2B chain | TUBB; TUBB2B | 2.06418101 |
| CON_ENSEMBLE | | | |
| NSBTAP00000025008; | | | |
| Q9BUF5; | | | |
| Q9H4B7 | | | |
| P05387 | 60S acidic ribosomal protein P2 | RPLP2 | 2.061830521 |
| P78371 | T-complex protein 1 subunit beta | CCT2 | 2.046453476 |
| Q9Y3B3 | Transmembrane emp24 domain-containing | TMED7 | 2.039887746 |
| | protein 7 | | |
| P60866 | 40S ribosomal protein S20 | RPS20 | 1.994560242 |
| P39656 | Dolichyl-diphosphooligosaccharide-protein | DDOST | 1.977687836 |
| | glycosyltransferase 48 kDa subunit | | |
| P28066 | Proteasome subunit alpha type-5 | PSMA5 | 1.94695727 |
| P50454 | Serpin H1 | SERPINH1 | 1.908407211 |
| P00558 | Phosphoglycerate kinase 1 | PGK1 | 1.899494171 |
| P24539 | ATP synthase F(0) complex subunit B1, | ATP5F1 | 1.889081955 |
| | mitochondrial | | |
| P02679 | Fibrinogen gamma chain | FGG | 1.881071726 |
| P12883 | Myosin-7 | MYH7 | 1.857381185 |
| P05556 | Integrin beta-1 | ITGB1 | 1.837862651 |
| P45880 | Voltage-dependent anion-selective channel | VDAC2 | 1.830989202 |
| | protein 2 | | |
| P02786 | Transferrin receptor protein 1; Transferrin | TFRC | 1.827234268 |
| | receptor protein 1, serum form | | |
| P55795; P31943 | Heterogeneous nuclear ribonucleoprotein | HNRNPH2; | 1.820849737 |
| | H2; Heterogeneous nuclear ribonucleoprotein | HNRNPH1 | |
| | H2, N-terminally processed; Heterogeneous | | |
| | nuclear ribonucleoprotein H; Heterogeneous | | |
| | nuclear ribonucleoprotein H, N-terminally | | |
| | processed | | |
| Q9UI42 | Carboxypeptidase A4 | CPA4 | 1.761526744 |
| O75390 | Citrate synthase, mitochondrial | CS | 1.720769882 |
| P68133; P68032; | Actin, alpha skeletal muscle; Actin, alpha | ACTA1; ACTC1; | 1.709612528 |
| P62736; P63267 | cardiac muscle 1; Actin, aortic smooth | ACTA2; ACTG2 | |
| | muscle; Actin, gamma-enteric smooth | | |
| | muscle | | |
| P24821 | Tenascin | TNC | 1.705941518 |
| P13533; A7E2Y1; | Myosin-6 | MYH6 | 1.676398595 |
| Q9H6N6 | | | |
| P63261; P60709; | Actin, cytoplasmic 2; Actin, cytoplasmic 2, | ACTG1; ACTB | 1.656216304 |
| Q562R1; A5A3E0 | N-terminally processed; Actin, cytoplasmic | | |
| | 1; Actin, cytoplasmic 1, N-terminally | | |
| | processed | | |
| P06576 | ATP synthase subunit beta, mitochondrial | ATP5B | 1.641290665 |
| Q5VTE0; P68104; | Putative elongation factor 1-alpha-like | EEF1A1P5; | 1.636025111 |
| Q05639 | 3; Elongation factor 1-alpha 1; Elongation | EEF1A1; | |
| | factor 1-alpha 2 | EEF1A2 | |
| Q9Y490; Q9Y4G6 | Talin-1 | TLN1 | 1.591281255 |
| P02452; P02458 | Collagen alpha-1(I) chain | COL1A1 | 1.589295705 |
| P69905 | Hemoglobin subunit alpha | HBA1 | 1.583057404 |
| P08758 | Annexin A5 | ANXA5 | 1.568888346 |
| P31949 | Protein S100-A11; Protein S100-A11, | S100A11 | 1.555873235 |
| | N-terminally processed | | |
| Q92930 | Ras-related protein Rab-8B | RAB8B | 1.547135671 |
| P23528; Q9Y281 | Cofilin-1 | CFL1 | 1.525413513 |
| P00738 | Haptoglobin; Haptoglobin alpha | HP | 1.504639943 |
| | chain; Haptoglobin beta chain | | |
| P62917 | 60S ribosomal protein L8 | RPL8 | 1.504460653 |
| P08865 | 40S ribosomal protein SA | RPSA | 1.504273097 |
| P15144 | Aminopeptidase N | ANPEP | 1.478000641 |
| P08133 | Annexin A6 | ANXA6 | 1.471323649 |
| P59666; P59665 | Neutrophil defensin 3; HP 3-56; Neutrophil | DEFA3; DEFA1 | 1.465414683 |
| | defensin 2; Neutrophil defensin 1; HP 1- | | |
| | 56; Neutrophil defensin 2 | | |
| P01834 | Ig kappa chain C region | IGKC | 1.457137426 |
| P11021 | 78 kDa glucose-regulated protein | HSPA5 | 1.448736827 |
| O94905; O75477 | Erlin-2; Erlin-1 | ERLIN2; ERLIN1 | 1.444409053 |
| Q16181 | Septin-7 | SEPTIN7 | 1.424034119 |
| P02751 | Fibronectin; Anastellin; Ugl-Y1; | FN1 | 1.415683746 |
| | Ugl-Y2; Ugl-Y3 | | |
| P68371; P04350 | Tubulin beta-4B chain; Tubulin beta-4A | TUBB4B; | 1.412516276 |
| | chain | TUBB4A | |
| Q14764 | Major vault protein | MVP | 1.403721491 |
| P04899; P63096; | Guanine nucleotide-binding protein G(i) | GNAI2; GNAI1; | 1.401363373 |
| P08754; P11488; | subunit alpha-2; Guanine nucleotide-binding | GNAI3 | |
| P09471; P19087; | protein G(i) subunit alpha-1; Guanine | | |
| A8MTJ3; P38405; | nucleotide-binding protein G(k) subunit | | |
| Q5JWF2 | alpha | | |
| Q15149 | Plectin | PLEC | 1.397479375 |
| O00159 | Unconventional myosin-Ic | MYO1C | 1.396870931 |
| P26038; P35241 | Moesin | MSN | 1.381834666 |
| P05026 | Sodium/potassium-transporting ATPase | ATP1B1 | 1.370250702 |
| | subunit beta-1 | | |
| Q9UJ83 | 2-hydroxyacy1-CoA lyase 1 | HACL1 | 1.365160624 |
| P30048 | Thioredoxin-dependent peroxide reductase, | PRDX3 | 1.361143748 |
| | mitochondrial | | |
| P42765 | 3-ketoacy1-CoA thiolase, mitochondrial | ACAA2 | 1.348441442 |
| Q16555 | Dihydropyrimidinase-related protein 2 | DPYSL2 | 1.334271749 |
| P13639; Q15029 | Elongation factor 2 | EEF2 | 1.329350789 |
| P04075 | Fructose-bisphosphate aldolase A | ALDOA | 1.323834737 |
| Q9NZT1 | Calmodulin-like protein 5 | CALML5 | 1.290933609 |
| P08473 | Neprilysin | MME | 1.273379008 |
| P01889 | HLA class I histocompatibility | HLA-B | 1.247062683 |
| | antigen, B-7 alpha chain | | |
| P00747 | Plasminogen; Plasmin heavy chain | PLG | 1.222115835 |
| | A; Activation peptide; Angiostatin; | | |
| | Plasmin heavy chain A, short form; | | |
| | Plasmin light chain B | | |
| P04843 | Dolichy1-diphosphooligosaccharide-protein | RPN1 | 1.203443527 |
| | glycosyltransferase subunit 1 | | |
| P05062 | Fructose-bisphosphate aldolase B | ALDOB | 1.181601206 |
| P14618 | Pyruvate kinase PKM | PKM | 1.156917572 |
| P18124 | 60S ribosomal protein L7 | RPL7 | 1.119624456 |
| P00734 | Prothrombin; Activation peptide fragment | F2 | 1.114295324 |
| | 1; Activation peptide fragment 2; Thrombin | | |
| | light chain; Thrombin heavy chain | | |
| P01040 | Cystatin-A; Cystatin-A, N-terminally | CSTA | 1.098798752 |
| | processed | | |
| P21796 | Voltage-dependent anion-selective | VDAC1 | 1.079421361 |
| | channel protein 1 | | |
| P42357 | Histidine ammonia-lyase | HAL | 1.068974813 |
| P04083 | Annexin A1 | ANXA1 | 1.060759226 |
| P23229 | Integrin alpha-6; Integrin alpha-6 heavy | ITGA6 | 1.05995369 |
| | chain; Integrin alpha-6 light chain; Processed | | |
| | integrin alpha-6 | | |
| P17844; Q92841; | Probable ATP-dependent RNA helicase | DDX5; DDX17; | 1.04998525 |
| O15523; O00571 | DDX5; Probable ATP-dependent RNA | DDX3Y; DDX3X | |
| | helicase DDX17; ATP-dependent RNA | | |
| | helicase DDX3Y; ATP-dependent RNA | | |
| | helicase DDX3X | | |
| P47929 | Galectin-7 | LGALS7 | 1.048348109 |
| P0DMV9; P0DMV8 | | HSPA1A | 1.030076345 |
| Q15582 | Transforming growth factor-beta-induced | TGFBI | 1.024225871 |
| | protein ig-h3 | | |
| P29992; O95837; | Guanine nucleotide-binding protein subunit | GNA11; GNA14; | 1.006320953 |
| P50148 | alpha-11; Guanine nucleotide-binding protein | GNAQ | |
| | subunit alpha-14; Guanine nucleotide- | | |
| | binding protein G(q) subunit alpha | | |
| P49327 | Fatty acid synthase; [Acyl-carrier-protein] S- | FASN | 0.999253591 |
| | acetyltransferase; [Acyl-carrier-protein] S- | | |
| | malonyltransferase; 3-oxoacyl-[acyl-carrier- | | |
| | protein] synthase; 3-oxoacyl-[acyl-carrier- | | |
| | protein] reductase; 3-hydroxyacyl-[acyl- | | |
| | carrier-protein] dehydratase; Enoyl-[acy]- | | |
| | carrier-protein] reductase; Oleoyl-[acyl- | | |
| | carrier-protein] hydrolase | | |
| P22735 | Protein-glutamine gamma- | TGM1 | 0.992776871 |
| | glutamyltransferase K | | |
| P63104 | 14-3-3 protein zeta/delta | YWHAZ | 0.973157883 |
| Q71DI3; Q16695; | Histone H3.2; Histone H3.1t; Histone | HIST2H3A; | 0.970722198 |
| P84243; P68431; | H3.3; Histone H3.1 | HIST3H3; | |
| Q6NXT2 | | H3F3A; | |
| | | HIST1H3A | |
| Q13835 | Plakophilin-1 | PKP1 | 0.96838824 |
| P07355; A6NMY6 | Annexin A2; Putative annexin | ANXA2 | 0.94677798 |
| | A2-like protein | | |
| Q09666 | Neuroblast differentiation- | AHNAK | 0.937266668 |
| | associated protein AHNAK | | |
| P35580 | Myosin-10 | MYH10 | 0.898457209 |
| P30101 | Protein disulfide-isomerase A3 | PDIA3 | 0.880051295 |
| P05546 | Heparin cofactor 2 | SERPIND1 | 0.866259893 |
| Q15493 | Regucalcin | RGN | 0.863951365 |
| P62987; P62979; | Ubiquitin-60S ribosomal protein | UBA52; RPS27A; | 0.855721792 |
| POCG47; POCG48; | L40; Ubiquitin; 60S ribosomal protein | UBB; UBC | |
| A0A2R8Y422 | L40; Ubiquitin-40S ribosomal protein | | |
| | S27a; Ubiquitin; 40S ribosomal protein | | |
| | S27a; Polyubiquitin- | | |
| | B; Ubiquitin; Polyubiquitin-C; Ubiquitin | | |
| P31944 | Caspase-14; Caspase-14 subunit | CASP14 | 0.852003098 |
| | p19; Caspase-14 subunit p10 | | |
| P06702 | Protein S100-A9 | S100A9 | 0.85128212 |
| Q92896 | Golgi apparatus protein 1 | GLG1 | 0.850999832 |
| P04406; O14556 | Glyceraldehyde-3-phosphate dehydrogenase | GAPDH | 0.84311231 |
| Q6UWP8 | Suprabasin | SBSN | 0.841398239 |
| P50995 | Annexin A11 | ANXA11 | 0.836142858 |
| Q13867 | Bleomycin hydrolase | BLMH | 0.794428507 |

TABLE 2
downregulated proteins

| | | | Intensity |
|---|---|---|---|
| Protein IDs | Protein names | Gene names | difference Log |
| O60701 | UDP-glucose 6-dehydrogenase | UGDH | −0.806416829 |
| Q99714 | 3-hydroxyacy1-CoA dehydrogenase type-2 | HSD17B10 | −0.858409882 |
| P31327 | Carbamoyl-phosphate | CPS1 | −0.859352748 |
| | synthase [ammonia], mitochondrial | | |
| P21980 | Protein-glutamine gamma- | TGM2 | −0.999712626 |
| | glutamyltransferase 2 | | |
| P00352 | Retinal dehydrogenase 1 | ALDH1A1 | −1.016107559 |
| Q99878; | Histone H2A type 1-J; | HIST1H2AJ; | −1.051007589 |
| Q96KK5; | Histone H2A type 1-H; | HIST1H2AH; | |
| Q9BTM1; | Histone H2A.J; | H2AFJ; | |
| Q16777; | Histone H2A type 2-C; | HIST2H2AC; | |
| Q6FI13; P20671; | Histone H2A type 2-A; | HIST2H2AA3; | |
| P0C0S8 | Histone H2A type 1-D; | HIST1H2AD; | |
| | Histone H2A type 1 | HIST1H2AG | |
| P52209 | 6-phosphogluconate dehydrogenase, | PGD | −1.075685501 |
| | decarboxylating | | |
| Q05682; | Caldesmon | CALD1 | −1.189534505 |
| REVQ13136; | | | |
| REVO75334 | | | |
| Q07954 | Prolow-density lipoprotein receptor-related | LRP1 | −1.193033854 |
| | protein 1; Low-density lipoprotein receptor- | | |
| | related protein 1 85 kDa subunit; Low-density | | |
| | lipoprotein receptor-related protein 1 515 kDa | | |
| | subunit; Low-density lipoprotein receptor- | | |
| | related protein 1 intracellular domain | | |
| P00367; P49448 | Glutamate dehydrogenase 1, | GLUD1; | −1.228942871 |
| | mitochondrial; Glutamate | GLUD2 | |
| | dehydrogenase 2, mitochondrial | | |
| P09525 | Annexin A4 | ANXA4 | −1.281707128 |
| P62807; O60814; | Histone H2B type 1-C/E/F/G/I; | HIST1H2BC; | −1.304754893 |
| P57053; Q99880; | Histone H2B type 1-K; | HIST1H2BK; | |
| Q99879; Q99877; | Histone H2B type F-S; | H2BFS; | |
| Q93079; Q5QNW6; | Histone H2B type 1-L; | HIST1H2BL; | |
| P58876; Q96A08; | Histone H2B type 1-M; | HIST1H2BM; | |
| A0A2R8Y619 | Histone H2B type 1-N; | HIST1H2BN; | |
| | Histone H2B type 1-H; | HIST1H2BH; | |
| | Histone H2B type 2-F; | HIST2H2BF; | |
| | Histone H2B type 1-D; | HIST1H2BD; | |
| | Histone H2B type 1-A | HIST1H2BA | |
| Q07065 | Cytoskeleton-associated protein 4 | CKAP4 | −1.51182429 |
| P02794 | Ferritin heavy chain; Ferritin | FTH1 | −1.625328064 |
| | heavy chain, N-terminally processed | | |
| QOUI17 | Dimethylglycine dehydrogenase, | DMGDH | −1.792697906 |
| | mitochondrial | | |
| P06396 | Gelsolin | GSN | −1.824964523 |
| P07195 | L-lactate dehydrogenase B chain | LDHB | −1.906670888 |
| P30613 | Pyruvate kinase PKLR | PKLR | −1.983826955 |
| Q8WZ42 | Titin | TTN | −2.049551646 |
| Q03135; P56539 | Caveolin-1 | CAV1 | −2.454488118 |
| Q6IQ26 | DENN domain-containing protein 5A | DENND5A | −2.668806076 |
| P24593 | Insulin-like growth factor-binding protein 5 | IGFBP5 | −3.568556468 |
| P17936 | Insulin-like growth factor-binding protein 3 | IGFBP3 | −3.728598277 |
| Q8NBJ4 | Golgi membrane protein 1 | GOLM1 | −6.706241608 |
| P49368 | T-complex protein 1 subunit gamma | CCT3 | −10.81299019 |

From the upregulated proteins identified at Table 1, the following relate to mechanical stimuli response, mechanical transduction machinery and cell response (e.g., integrin, cadherin, cytoskeleton remodeling), muscle related proteins and endocytosis. These proteins include:
PREX1 (Phosphatidylinositol 3,4,5-trisphosphate-dependent Rac exchanger 1 protein);
ITGB1 (Integrin beta-1);
TLN1 (Talin-1);
VCL (Vinculin);
FSCN1 (Fascin);
VTN (Vitronectin);
FLNA (Filamin-A);
ACTN1; ACTN4 (Alpha-actinin-1; Alpha-actinin-4);
TUBA1B; TUBA1C; TUBA1A; TUBA3E (Tubulin alpha-1B chain; Tubulin alpha-1C chain; Tubulin alpha-1A chain; Tubulin alpha-3E chain);
TUBB; TUBB2B (Tubulin beta chain; Tubulin beta-2B chain);
ACTA1; ACTC1; ACTA2; ACTG2 (Actin);
ACTG1; ACTB (Actin, cytoplasmic 2; Actin, cytoplasmic 2,N-terminally processed; Actin, cytoplasmic 1; Actin, cytoplasmic 1,N-terminally processed);
MYL4 (Myosin light chain 4);
MYL6; MYL6B (Myosin light polypeptide 6; Myosin light chain6 B);
MYH6 (Myosin-6);
RAB11A; RAB11B (Ras-related protein Rab-11A; Ras-related protein Rab-11B); and
S100A11 (Protein S100-A11; Protein S100-A11, N-terminally processed).

1-39. (canceled)

40. A method for producing extracellular vesicles (EVs) from muscle cells, the method comprising:
a) providing at least one three-dimensional (3D) scaffold comprising:
(i) a plurality of layers, wherein each layer of the plurality of layers comprises a plurality of elastic microfibers spaced from each other, wherein each microfiber of the plurality of elastic microfibers extends from a first end of the at least one 3D scaffold towards a second end of the at least one 3D scaffold, wherein each of the plurality of elastic microfibers is aligned along and/or in parallel to a longitudinal axis, and wherein the plurality of layers are stacked one on top of the other; and
(ii) a plurality of spacers, wherein each spacer of the plurality of spacers is disposed between consecutive layers of the plurality of layers, thereby spacing therebetween;
b) seeding and culturing a population of muscle cells on and/or within the at least one 3D scaffold of act (a), thereby enabling the formation of a 3D multi-layer muscle fibers structure thereon; and
c) applying at least one dynamic mechanical loading stimulation to the at least one 3D scaffold comprising muscle fibers of act (b), thereby affecting the production and/or secretion of extracellular vesicles (EVs) from the 3D multi-layer structure of muscle cells cultured thereon into a medium.

41. The method according to claim 40, wherein each spacer of the plurality of spacers within the at least one 3D scaffold comprises a plurality of elongated spacing members, such that consecutive layers are spaced by the plurality of spacing members, wherein the plurality of spacing members are spaced apart in parallel from each other between consecutive layers, and wherein each of the plurality of spacing members is extending along a direction perpendicular to the longitudinal axis.

42. The method according to claim 41, wherein the plurality of spacing members within the at least one 3D scaffold comprise a first plurality and a second plurality, such that consecutive layers are spaced by said first and second pluralities of spacing members, wherein the first plurality of the parallel spacing members are disposed in the vicinity of the first end of the at least one 3D scaffold, wherein the second plurality thereof are disposed in the vicinity of the second end of the at least one 3D scaffold, and wherein the first and second pluralities of spacing members define an empty space therebetween.

43. The method according to claim 40, wherein each microfiber of the plurality of elastic microfibers comprises one or more synthetic or natural polymers selected from at least one of polyacrylamide, polydimethylsiloxane (PDMS), polypropylene (PP), polylactic acid (PLA), poly-L-lactic acid (PLLA), poly(lactic-co-glycolic) acid (PLGA), polyacrylate, poly(vinyl alcohol), poly(ethylene glycol), polycaprolactone (PCL), cellulose, silk, alginate, fibrin, gelatin, collagen, hyaluronic acid (HA), chitosan, dextran, or copolymers thereof.

44. The method according to claim 40, wherein each microfiber of the plurality of elastic microfibers has a diameter in a range of about 10 μm to about 1000 μm.

45. The method according to claim 40, wherein the at least one 3D scaffold is configured to be stretched and to undergo more than about 10% strain without reaching the yield point thereof; or wherein the at least one 3D scaffold has a Young's modulus in a range of 0.1 to about 2 MPa.

46. The method according to claim 40, wherein act (a) further comprises providing a bioreactor system comprising at least one main chamber and a medium disposed therein, wherein the method comprises inserting the at least one 3D scaffold into the main chamber prior to act (c), wherein the at least one main chamber accommodates therein at least two opposing platforms, wherein inserting the at least one 3D scaffold into the main chamber comprises coupling said two opposing platforms to opposing portions of the at least one 3D scaffold, such that the at least one 3D scaffold is extending therebetween, and wherein act (b) is performed within the main chamber.

47. The method according to claim 46, wherein act (c) comprises displacing at least one of the two opposing platforms within the main chamber, thereby inducing mechanical loading stimulations on the at least one 3D scaffold extending therebetween, wherein the mechanical loading stimulations are selected from the group consisting of compression, tension, torsion, bending, and combinations thereof.

48. The method according to claim 47, wherein the two opposing platforms are coupled to opposing portions of the at least one 3D scaffold, such that the plurality of elastic microfibers are aligned along and/or in parallel to the longitudinal axis, wherein act (c) comprises displacing the two opposing platforms away from each other along the longitudinal axis, thereby inducing tension to the at least one 3D scaffold extending therebetween.

49. The method according to claim 48, wherein act (c) comprises displacing the two opposing platforms away and towards each other repeatedly, thereby inducing repeating tension cycles to the at least one 3D scaffold, at a certain frequency, for a certain time duration.

50. The method according to claim 49, wherein the certain frequency is in a range of about 0.1 to about 5 Hz.

51. The method according to claim 40, wherein the extracellular vesicles are selected from the group consisting of exosomes, microvesicles, apoptotic bodies, ectosomes, and combinations thereof.

52. The method according to claim 51, wherein the extracellular vesicles are exosomes.

53. The method according to claim 40, wherein the muscle cells are mammalian muscle cells.

54. The method according to claim 52, wherein the mammalian muscle cells are human muscle cells selected from the group consisting of human skeletal muscle cells (SkMCs), human induced pluripotent stem cells derived-cardiomyocytes, human smooth muscle cells, and a combination thereof.

55. The method according to claim 40, wherein the method further comprises act (d) of collecting the medium of act (c) and act (e) of isolating the secreted extracellular vesicles dispersed within the medium of act (d).

56. Extracellular vesicles produced by the method of claim 40, characterized by expressing at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof, wherein the expression of the at least one protein is upregulated compared to extracellular vesicles produced without being subjected to at least one mechanical loading stimulation.

57. Extracellular vesicles secreted from muscle cells, characterized by expressing at least one marker selected from CD9, CD63, and CD81, and expressing in an upregulated amount compared to extracellular vesicles produced without being subjected to at least one mechanical loading stimulation, at least one protein selected from the group consisting of: PREX1, ITGB1, TLN1, VCL, FSCN1, VTN, FLNA, ACTN1, ACTN4, TUBA1C, TUBA1A, TUBA3E, TUBA1B, TUBB, TUBB2B, ACTA1, ACTC1, ACTA2, ACTG2, ACTG1, ACTB, MYL4, MYL6, MYL6B, MYH6, RAB11A, RAB11B, S100A11, and combinations thereof.

58. A composition comprising the extracellular vesicles of claim 56.

59. The composition according to claim 58, wherein the extracellular vesicles are exosomes.

60. A method of prevention or treatment of a disease or disorder, the method comprising:
administering to a subject in need thereof the composition according to claim 58, wherein the disease or disorder is selected from the group consisting of blood vessel diseases, cardiac diseases, skeletal muscle diseases, and combinations thereof.

61. A three-dimensional (3D) scaffold configured to support a population of muscle cells seeded and cultured thereon, the 3D scaffold comprising:
a plurality of layers, each layer of the plurality of layers comprises a plurality of elastic microfibers spaced apart from each other, wherein each microfiber of the plurality of elastic microfibers extends from a first end of the 3D scaffold towards a second end of the 3D scaffold; wherein the plurality of elastic microfibers are aligned along and/or in parallel to a longitudinal axis and to each other; and wherein the plurality of layers are stacked one on top of the other; and
a plurality of spacers, each spacer of the plurality of spacers is disposed between consecutive layers of the plurality of layers, thereby spacing therebetween;
wherein the 3D scaffold is configured to support an expansion of a population of muscle cells into a 3D multi-layer structure of muscle fibers.
